(19) United States
(12) Patent Application Publication
(10) Pub. No.: US 2025/0066790 A1
(43) Pub. Date: Feb. 27, 2025

(54) ANTISENSE OLIGONUCLEOTIDES FOR MODIFYING PROTEIN EXPRESSION

(71) Applicant: University of Rochester, Rochester, NY (US)

(72) Inventors: Peng YAO, Pittsford, NY (US); Omar HEDAYA, Rochester, NY (US)

(21) Appl. No.: 18/708,838

(22) PCT Filed: Nov. 7, 2022
(86) PCT No.: PCT/US2022/049117

§ 371 Date: May 9, 2024

Related U.S. Application Data
(60) Provisional application No. 63/263,853, filed on Nov. 10, 2021.
(60) Provisional application No. 63/373,792, filed on Aug. 29, 2022.

(51) Int. Cl. C12N 15/113

(52) U.S. Cl. CPC C12N 15/1136; C12N 2310/11; C12N 2310/313; C12N 2310/321; C12N 2310/531

(57) ABSTRACT

The present application provides antisense oligonucleotides capable of modulating translation of a main open reading frame in a mRNA of a target gene. Also provided are method of making and method of use for such oligonucleotides.

This application claims priority from U.S. Provisional Application No. 63/263,853, filed Nov. 10, 2021, and U.S. Provisional Application No. 63/373,792, filed Aug. 29, 2022. The entirety of all the aforementioned applications is incorporated herein by reference.

This invention was made with government support under HL132899 and HL147954 awarded by the National Institutes of Health. The government has certain rights in the invention.

### FIELD

This application relates to the field of protein translation. More specifically, the present invention provides compositions and methods useful for endogenously increasing or decreasing translation, especially in the context of treatments for diseases.

### BACKGROUND

In many disease conditions it would be beneficial to enhance or suppress the production of one or more proteins. In one approach, RNA interference (RNAi) agents, such as siRNAs, are delivered to cells to suppress protein expression. In another approach, exogenous protein coding expression vectors are delivered to cells to enhance protein expression. However, these approaches have limitations. For example, the development of siRNA-based drugs for therapeutic use suffers from low efficiency of siRNA delivery to target cells and the degradation of siRNAs by nucleases in biological fluids. Secondly, gene therapy approaches often involve packaging whole genes in a virus, such as adeno-associated viruses (AAV), which can be susceptible to size constraints, as well as complications, such as immunogenicity. Accordingly, there is a need for new approaches for increasing or decreasing protein translation, especially in the context of treating diseases.

The present application addresses the foregoing limitations and provides novel strategies for the design and use of antisense oligonucleotides (ASOs) to selectively increase or decrease translation of proteins from their endogenous genes.

### SUMMARY

One aspect of the present application relates to an antisense oligonucleotide (ASO) that comprises 8-50 nucleotide, including one or more modified nucleotides, wherein the ASO is capable of binding to a target region in an upstream open reading frame (uORF) of a mRNA of a target gene and wherein the target region forms a double-stranded stem structure with a region of the uORF that is downstream of, and adjacent to, the start codon of the uORF. The binding of the ASO to the target region disrupts the double-stranded stem structure of the uORF and enhances translation of a main open reading frame (mORF) downstream of the uORF of the mRNA of the target gene.

Another aspect of the present application relates to an ASO comprising 8-50 nucleotides, including one or more modified nucleotides, wherein the ASO is capable of binding to a target region in an uORF of a mRNA of a target gene and wherein the target region is downstream of, and adjacent to, a start codon of the uORF. The binding of the ASO to the target region forms a double-stranded ASO/mRNA hybrid structure that inhibits translation of a downstream mORF of the mRNA of the target gene.

Another aspect of the present application relates to an ASO comprising 8-50 nucleotides, including one or more modified nucleotides, wherein the ASO is capable of binding to a target region in an mORF of a mRNA of a target gene and wherein the target region is downstream of, and adjacent to, a start codon of the mORF. The binding of the ASO to the target region forms a double-stranded ASO/mRNA hybrid structure that enhances translation of the mORF of the mRNA of the target gene.

Another aspect of the present application relates to a pharmaceutical composition comprising an ASO of the present application and a pharmaceutically acceptable carrier.

Another aspect of the present application relates to a method for increasing or decreasing translation of a target protein in a subject. The method comprises the step of administering to the subject an effective amount of the pharmaceutical composition of the present application.

### BRIEF DESCRIPTION OF THE DRAWINGS

FIG. 1, Panels A-C show models for translational activation or suppression. Panel A shows a model for type I uORF ASO-targeted suppression of uORF translation and activation of mORF translation. Panel B shows a model for type II uORF ASO-targeted activation of uORF translation and suppression of mORF translation. Although a dsRNA stem structure is shown in the figure, this type of ASOs does not require the presence of such structure to form a an ASO-mRNA hybrid region. Panel C shows a model for Type II mORF ASO-targeted activation of mORF translation.

FIG. 2, Panels A-D show a composite of drawings and pictures showing crosstalk between uORF and an adjacent double-stranded RNA structural element in translational regulation of mORF translation. Panel A shows a schematic of the dual luciferase reporter assay. Panel B shows a schematic of FLuc reporter constructs. Panel C shows results of dual luciferase reporter assay using a series of constructs that contain uORF start codon and adjacent dsRNA structure (Kan-HP1 hairpin) that is located at different distances. N=3 biological replicates. Data were presented as mean±SEM. P values were calculated by unpaired two-tailed Student t test. Panel D shows results of dual luciferase reporter assay using mutant construct that contains three nucleotide mutations leading to disrupted stem structure. N=3 biological replicates. Data were presented as mean±SEM. P values were calculated by unpaired two-tailed Student t test. No AUG: ATG-to-TTG mutation. AUG −2: start codon is located at −2 position relative to the hairpin. Intact: stable WT hairpin. Weakened: three mutations introduced in the hairpin to destabilize the structure.

FIG. 3 shows localization of artificial uORF-KanHP1 mRNA variants in 40S ribosomal subunit or 80S monosome fractions in HEK293T lysates upon 10-35% sucrose gradient centrifugation. Experiments were repeated 2 times, and representative data were shown.

FIG. 4, Panels A-D show a composite of drawings and pictures identifying uORFs as major translational regulatory elements in mRNAs encoding cardiac transcription factors. Panel A illustrates the overlap of mRNAs containing uORFs based on ribosome profiling (Ribo-Seq) in human and mouse failing hearts along with an ontological analysis of human cardiac uORFs. Multiple cardiac mRNAs and embedded uORFs are highlighted such as GATA4. Panel B shows that GATA4 uORFs are present across mammals as shown in a representative group of species. Panel C shows schematic of WT and mutant GATA4 5′ UTR cloned in FLuc reporter constructs. Panel D shows that dsRNA element is required for uORF-mediated translational repression of mORF. Dual luciferase reporter assay with WT, ΔuORF, secondary structure mutant, and rescuing mutant. N=3 biological replicates. Data were presented as mean±SEM. P values were calculated by unpaired two-tailed Student t test.

FIG. 5, Panels A-F show a composite of drawings and pictures showing a mechanism-based design of ASOs for regulating mORF translation. Panels A-B, left show a schematic of designed ASOs targeting the GATA4 uORF dsRNA clement. Panels A-B, middle show dual luciferase reporter assays with WT and ΔuORF mutant GATA4 after transfection of type I uotASO (ASO1) and type II uotASO (ASO2) (oligo sequences are shown in Panel F). N=3 biological replicates. Comparisons were performed by unpaired two-tailed Student t test. Panel C shows Western blot analysis of dose-responsive manipulation of endogenous GATA4 protein expression by ASO1 and ASO2 in AC16 human cardiomyocyte cell line. Panels D-E show polysome profiling of WT and ΔuORF cells with ASO1/ASO2 treatment in AC16 cells. Panel F shows α-β actin immunostaining of AC16 cells after transfection of control ASO, ASO1 and ASO2. Cell surface area was measured and quantified (n≥200 cells). Scale bar: 20 mm. In the violin plot, the solid line shows median value for the group and dashed lines represent two quartile lines in each group. P values were calculated by unpaired two-tailed Student t test.

FIG. 6, Panels A-G show a composite of drawings and pictures showing therapeutic treatment in an ISO-induced cardiac hypertrophy model using GATA4 ASO2. Panel A shows a schematic of ASO treatment of ISO-induced cardiac hypertrophy mouse model. Panel B shows WGA staining of hearts of ISO treated mice. Panel C shows HW/TL ratio of hearts of ISO treated mice. N=6 for Ctrl ASO groups; N=7 for GATA4 ASO groups (males were indicated by black dots and females as pink dots). Panel D shows quantification of CM cell size. Panel E shows Western blot analysis of GATA4 protein expression in the hearts. N=6 for Ctrl ASO groups; N=7 for GATA4 ASO groups (males were indicated by black dots and females as pink dots). Panel F shows RT-qPCR measurement of GATA4 mRNA expression in the hearts. N=6 for Ctrl ASO groups; N=7 for GATA4 ASO groups (males were indicated by black dots and females as pink dots). Panel G shows RT-qPCR measurement of hypertrophy marker gene Nppa in mouse heart samples. N=6 for Ctrl ASO groups; N=7 for GATA4 ASO groups (males were indicated by black dots and females as pink dots). Data were presented as mean±SEM. P values were calculated by two-way ANOVA with Tukey's multiple comparisons test (C-G).

FIG. 7, Panels A-H show a composite of drawings and pictures showing therapeutic treatment in a TAC-induced cardiac hypertrophy model using GATA4 ASO2. Panel A is a schematic of TAC surgery-induced cardiac hypertrophy mouse model. Panel B shows HW/TL ratio of hearts of mice under TAC surgery. N=10 for Ctrl ASO group; N=12 for GATA4 ASO group (males were indicated by black dots and females as pink dots). Panel C shows wheat germ agglutinin (WGA) straining for CM hypertrophy. Cell surface arca was measured and quantified (n≥200 cells). Scale bar: 50 um. In the violin plot, solid line shows median value for the group and dashed lines represent two quartile lines in each group. P values were calculated by unpaired two-tailed Student t test. Panel D shows picrosirius red staining for collagen deposition during cardiac fibrosis. Scale bar: 1 mm. P values were calculated by unpaired two-tailed Student t test. Panel E shows EF and FS measurement by echocardiography for cardiac function. N=10 for Ctrl ASO group and N=13 for GATA4 ASO. Panel F shows Western blot analysis of GATA4 protein expression in the hearts. N=10 for Ctrl ASO group; N=12 for GATA4 ASO group. Panel G shows RT-qPCR measurement of GATA4 mRNA expression in the hearts. N=10 for Ctrl ASO group; N=12 for G GATA4 ASO group. Panel H shows RT-qPCR measurement of hypertrophy marker gene Nppa in mouse heart samples. N=10 for Ctrl ASO group; N=12 for GATA4 ASO group. Data were presented as mean±SEM. P values were calculated by unpaired two-tailed Student t test.

FIG. 8 is a composite of drawings and pictures showing a model illustrating a a mechanism-based design of GATA4-inhibitory ASOs for treatment of cardiac hypertrophy and a general concept for manipulating uORF activity to achieve bidirectional control of mORF translation using ASO drugs.

FIG. 9, Panels A-E show the design and use of type II mot ASOs to increase translation from mORF. Panel A shows type II motASO target sequences in the mRNA sequences corresponding to the mORFs or eIF4G2, TBX5 and GATA4. Panel B is a Western blot showing increased translation of eIF4G2 mORF relative to a β-actin internal control. Panel C is a Western blot showing increased translation of TBX5 mORF relative to a β-actin internal control. Panel D is a Western blot showing increased translation of GATA4 mORF relative to a β-actin internal control. Panel E shows a model for type II motASO-targeted translational activation.

FIG. 10, Panels A-C show the effects of introducing mutations into the uORF start codons and dsRNA regions of the TBX5 5′ UTR. Panel A shows the putative dsRNA structure (TurboFold) near the uORF start codon, including constructs of a WT and a structure-disrupting mutant (Mut) containing mutations introduced the stem region to weaken the dsRNA structure. Panel B shows the results of a dual luciferase (DLR) assay to evaluate the effects of the foregoing mutations, including a double mutant (Mut+ΔuORF (which contains a defective uORF start site)) on translation of the TBX5 mORF in human HEK293T cells. Panel C shows a Western blot showing the effects on mORF translation of DDX3X, GATA4, NKX2-5, TBX5, MEF2C, and ACTB (β-actin) following transfection of an siRNA against DDX3X (i.e. knockdown) in human AC16 cardiomyocyte cells.

FIG. 11 shows a predicted secondary structure in the 5′ UTR of TBX5.

FIG. 12 shows a predicted secondary structure in the 5′ UTR of TBX20.

FIG. 13 shows a predicted secondary structure in the 5′ UTR of GATA6.

FIG. 14 shows a predicted secondary structure in the 5′ UTR of MYOCD.

FIG. 15, Panel A-B shows on-target and off-target effects of GATA4-targeting ASO. Panel A shows in vitro RNA SHAPE analysis of the secondary structure of GATA4 uORF-dsRNA region under ASO1 and ASO2 treatment. Panel B shows that GATA4 ASO2 does not cause any transcriptome-wide mRNA degradation. AC16 human CMs transfected with ASO2 at baseline were subject to RNA-seq (n=3). (Panel C) RT-qPCR measurement of off-target mRNAs or long non-coding RNA (lncRNA) for ASO2.

FIG. 16, Panels A-B show ASO-mediated modulation of uORF regulates GATA4 protcin expression and CM hypertrophy in human ESC-derived CMs. Panel A shows: Left and middle: Western blot analysis of GATA4 protein expression in ESC-derived CMs by ASO1 and ASO2 (50 nM) treatment. Right: RT-qPCR measurement of GATA4 mRNA normalized to ACTB. Panel B shows: Representative images of α-Actinin (green) and NKX2-5 (red) immunostaining in addition to DAPI (bluc) in ESC-derived CMs treated with control ASO, ASO1, or ASO2. Scale bar: 50 mm. Co-staining of α-Actinin (green) and NKX2-5 (red) discerns CMs from misdifferentiated cells. Cell surface area was measured for five different clumps of cells as the total surface area was divided by the number of cells. Data are represented as mean±SD. * P<0.05, ** P<0.01, *** P<0.001; Statistical significance was confirmed by unpaired two-tailed Student t test for B (N=3 biological replicates) and by ANOVA followed by Holm-Sidak post hoc test for A (N=3 biological replicates).

FIG. 17, Panels A-E show ASO-mediated modulation of uORF regulates GATA4 protein expression and CM hypertrophy in human ESC-derived CMs. Panel A shows the translation-inhibiting effects of different lengths of 2′-O-methyl modified GATA4 uORF-enhancing ASO2 (SEQ ID NO:7) in AC16 cells transfected with 50 nM ASO for 24 hours. The assay was repeated twice and representative data were shown. Panel B shows the schematic of various ASOs with different chemical modifications used in this study. Panel C-F show the enhancement of the uORF-targeting ASO2 through various chemistries. ASO2 with locked nucleic acid bases (LNA, SEQ ID NO:57) produced greater suppression of GATA4 protein levels compared to 2′-O-methylated (SEQ ID NO:7), 2′-O-methoxy-ethyled (MOE, SEQ ID NO:56), and phosphorothioate (PS, SEQ ID NO:58) backbone (Panel C). Panel E shows the combination of 2′-O-methyl and LNA is superior to 2′-O-methyl alone. Panel D, Panel F show RT-qPCR measurement of GATA4 mRNA in Panel A or Panel C with Actb mRNA used as a normalizer shows no changes in mRNA levels.

FIG. 18 shows reversal treatment of ISO-induced cardiac hypertrophy model. Panel A is a schematic of GATA4 ASO2 reversal model treating ISO-induced cardiac hypertrophy in WT mice. Panel B shows HW/TL ratio of WT mice injected with ISO for 40 days and 4 injections of GATA4 ASO2. N=5/6/5 (male mice). Panel C shows WGA staining of the surface area of CMs (n>400). Panel D shows the results of alanine aminotransferase assay for serum samples. Data were shown as mean±SD. P values: unpaired two-tailed Student t test. **** P<0.0001.

FIG. 19 shows a generalization of uORF- and mORF-targeted ASOs for other mRNAs encoding transcription and translation factors. (A) Western blot analysis of the three target proteins upon transfection of 50 nM Type II uORF-enhancing ASOs with modifications for mRNAs of MEF2C, NKX2-5, and eIF4G2. (B-D) Testing ASO Type III main open reading frame (mORF)-enhancing ASOs. (B) GATA4 targeting ASOs enhance its protein levels. Like with uORF-specific ASOs, the combination of 2′-O-methyl and LNA is superior to 2′-O-methyl alone and does not change mRNA levels (C). 2′-O-methyl and LNA mORF ASOs (50 nM) increase the protein levels of mRNA with cognate start codons in the case of NKX2-5 and MEF2C (D). Data are represented as mean±SD. * P<0.05, ** P<0.01, ** P<0.001. Statistical significance was confirmed by an unpaired two-tailed Student t test for A-D (N=3 biological replicates).

FIG. 20 shows a list of sequences described in the present application.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting, and the scope of the application is intended to encompass all equivalents, alternatives, and modifications, either generally known, or incorporated here. The described aspects, features, advantages, and characteristics of the invention may be combined in any suitable manner in one or more further embodiments. One skilled in the relevant art will recognize that the invention may be practiced without one or more of the specific aspects or advantages of a particular embodiment. In other instances, additional aspects, features, and advantages may be recognized and claimed in certain embodiments that may not be present in all embodiments of the invention. Further, one skilled in the art will recognize many techniques and materials similar or equivalent to those described here, which could be used in the practice of the aspects and embodiments of the present application. The described aspects and embodiments of the application are not limited to the methods and materials described.

Unless otherwise defined, all technical and scientific terms used herein have the same meaning as commonly understood by one of ordinary skill in the art to which this application belongs.

Ranges may be expressed herein as from “about” one particular value and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

As used in this specification and the appended claims, the singular forms “a,” “an” and “the” include plural referents unless the content clearly dictates otherwise. Thus, for example, reference to “a peptide” includes “one or more” peptides or a “plurality” of such peptides.

### I. DEFINITIONS

As used herein, the following terms or phrases (in parentheses) shall have the following meanings:

A “target protein” refers to a protein that one desires to increase or decrease in amount, concentration, or activity. In certain embodiments, the target protein is encoded by the primary open reading frame of a target transcript.

A “main open reading frame” or “mORF” refers to the portion of the target transcript that encodes the main (or primary) protein associated with an mRNA transcript. In certain embodiments, the mORF encodes the target protein.

The terms “uORF” and “upstream open reading frame” refer to a portion of a target transcript that comprises a start site upstream of (i.e. 5′ of) the mORF and an in frame termination codon. In certain embodiments, a uORF is the portion of the target transcript that is translated when translation is initiated at a uORF start site. In certain embodiments, a uORF does not overlap with an mORF. In certain embodiments, a uORF overlaps with the mORF. In certain embodiments a uORF overlaps with another uORF. In certain embodiments, a uORF is out of frame with an mORF.

The term “oligonucleotide” refers to a compound comprising a plurality of linked nucleosides. In certain embodiments, an oligonucleotide comprises one or more unmodified ribonucleosides and/or unmodified deoxyribonucleosides and/or one or more modified nucleosides.

The term “oligonucleoside” refers to an oligonucleotide in which none of the internucleoside linkages contains a phosphorus atom. The term oligonucleotides include oligonucleosides.

The term “internucleoside linkage” refers to a covalent linkage between adjacent nucleosides in an oligonucleotide.

A “naturally occurring internucleoside linkage” refers to a 3′ to 5′ phosphodiester linkage.

The term “modified internucleoside linkage” refers to any internucleoside linkage other than a naturally occurring internucleoside linkage.

The terms “antisense oligonucleotide” or “ASO” refer to a compound comprising or consisting of an oligonucleotide or modified oligonucleotide at least a portion of which is complementary to a target nucleic acid, a target nucleotide sequence (target sequence), a target site of a nucleotide sequence (target site), or a target region of a nucleotide sequence (target region), to which it is capable of hybridizing, resulting in at least one antisense activity. In some embodiments, the ASO comprises a nucleotide sequence that is at least 50%, 60%, 70%, 80%, 90% or 95% complementary to the target sequence, the target site or the target region. In some embodiments, an ASO comprises an antisense oligonucleotide conjugated to a conjugate group. In some embodiments, the conjugate group is a non-nucleotide conjugate group.

An “antisense activity” refers to any detectable and/or measurable change attributable to the hybridization of an antisense oligonucleotide to its target nucleic acid, target sequence, target site or target region.

The term “GATA4 transcript” refers to a native GATA4 mRNA transcript encoding an upstream open reading frame (uORF) and a main open reading frame (mORF) encoding the GATA4 protein.

The term “nucleoside” refers to a molecule comprising a nucleobase moiety such as a purine or pyrimidine base covalently linked to a sugar moiety such as ribose or deoxyribose sugar. Nucleosides include, but are not limited to, naturally occurring nucleosides (as found in DNA and RNA) and modified nucleosides. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). Nucleosides may be linked to a phosphate moiety.

The term “modified nucleoside” refers to a nucleoside comprising at least one chemical modification compared to naturally occurring RNA or DNA nucleosides. Modified nucleosides comprise a modified sugar moiety and/or a modified nucleobase.

The term “nucleotide” refers to a nucleoside having one or more phosphate groups joined in ester linkages to the sugar moiety. Exemplary nucleotides include nucleoside monophosphates, diphosphates and triphosphates.” The term “linked nucleosides” are nucleosides that are connected in a continuous sequence (i.e., no additional nucleosides are present between those that are linked).

The term “modified nucleotide” refers to a nucleotide comprising a modified nucleoside with optional modifications in the phosphate linking group. In certain exemplary embodiments, the modified nucleotide is modified at any position so as to alter certain chemical properties of the nucleotide yet retain the ability of the modified nucleotide to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotides such as those described in Herdewijn, Antisense Nucleic Acid Drug Dev., 2000 Aug. 10(4):297-310. The modified nucleotide may also comprise modifications to the sugar moiety of the nucleotides. For example, the 2′ OH-group may be replaced by a group selected from H, OR, R, F, Cl, Br, I, SH, SR, NH2, NHR, NR2, COOR, or OR, wherein R is substituted or unsubstituted C1-C6 alkyl, alkenyl, alkynyl, aryl, etc. Other possible modifications include those described in U.S. Pat. Nos. 5,858,988, and 6,291,438. The phosphate linking group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5):317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said modified nucleotides in vivo or in vitro.

The term “chemical modification” refers to a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

The term “furanosyl” refers to a structure comprising a 5-membered ring comprising four carbon atoms and one oxygen atom.

The phrase “naturally occurring sugar moiety” refers to a ribofuranosyl as found in naturally occurring RNA or a deoxyribofuranosyl as found in naturally occurring DNA.

The term “sugar moiety” refers to a naturally occurring sugar moiety or a modified sugar moiety of a nucleoside.

The term “modified sugar moiety” refers to a substituted sugar moiety or a sugar surrogate.

The term “substituted sugar moiety” refers to a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

The term “2′-substituted sugar moiety” refers to a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

The term “2′-F nucleoside” refers to a nucleoside comprising a sugar comprising fluoroine at the 2′ position. Unless otherwise indicated, the fluorine in a 2′-F nucleoside is in the ribo position (replacing the OH of a natural ribose).

The term “2′-(ara)-F” refers to a 2′-F substituted nucleoside, wherein the fluoro group is in the arabino position.

The term “sugar surrogate” refers to a structure that does not comprise a furanosyl and is capable of replacing the naturally occurring sugar moiety of a nucleoside, such that the resulting nucleoside sub-units are capable of linking together and/or linking to other nucleosides to form an oligonucleotide which is capable of hybridizing to a complementary oligonucleotide. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

The term “bicyclic sugar moiety” refers to a modified sugar moiety comprising a 4 to 7 membered ring (including but not limited to a furanosyl) comprising a bridge connecting two atoms of the 4 to 7 membered-ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

The term “nucleobase” refers to a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

The terms, “unmodified nucleobase” and “naturally occurring nucleobase” refer to the naturally occurring heterocyclic nucleobases of RNA or DNA: the purine bases adenine (A) and guanine (G), and the pyrimidine bases thymine (T), cytosine (C) (including 5-methyl C), and uracil (U).

The term “modified nucleobase” refers to any nucleobase that is not a naturally occurring nucleobase.

The term “bicyclic nucleoside” or “BNA” refers to a nucleoside comprising a bicyclic sugar moiety.

The terms “constrained ethyl nucleoside” and “cEt” refer to a nucleoside comprising a bicyclic sugar moiety comprising a 4′-CH(CH₃)—O-2′bridge.

The term “locked nucleic acid nucleoside” or “LNA” refers to a nucleoside comprising a bicyclic sugar moicty comprising a 4′-CH₂—O-2′bridge.

The term “2′-substituted nucleoside” refers to a nucleoside comprising a substituent at the 2′-position other than H or OH. Unless otherwise indicated, a 2′-substituted nucleoside is not a bicyclic nucleoside.

The term “2′-deoxynucleoside” refers to a nucleoside comprising 2′-H furanosyl sugar moicty, as found in naturally occurring deoxyribonucleosides (DNA). In certain embodiments, a 2′-deoxynucleoside may comprise a modified nucleobase or may comprise an RNA nucleobase (e.g., uracil).

The term “oligomeric compound” refers to a polymeric structure comprising two or more sub-structures. In certain embodiments, the sub-structures are nucleotides or nucleosides. In certain embodiments, an oligomeric compound comprises an oligonucleotide. In certain embodiments, an oligomeric compound consists of an oligonucleotide. In certain embodiments, an oligomeric compound consists of an antisense oligonucleotide.

The term “terminal group” refers to one or more atom attached to either, or both, the 3′ end or the 5′ end of an oligonucleotide. In certain embodiments a terminal group is a conjugate group. In certain embodiments, a terminal group comprises one or more terminal group nucleosides.

The term “conjugate group” refers to an atom or group of atoms bound to an oligonucleotide or oligomeric compound. In general, conjugate groups modify one or more properties of the oligonucleotide or oligomeric compound to which they are attached, including, but not limited to pharmacodynamic, pharmacokinetic, binding, absorption, cellular distribution, cellular uptake, charge and/or clearance properties.

The term “conjugate linking group” refers to any atom or group of atoms used to attach a conjugate to an oligonucleotide or oligomeric compound.

The terms “detecting” and “measuring” refer to that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

The phrase “detectable and/or measurable activity” refers to a measurable activity that is not zero.

The term “essentially unchanged” refers to little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

The term “expression” refers to the process by which a gene ultimately results in a protein. Expression includes, but is not limited to, transcription, post-transcriptional modification (e.g., splicing, polyadenylation, addition of 5′-cap), translation, and post-translational modification.

The term “translation” refers to the process in which a polypeptide (e.g. a protein) is translated from an mRNA. In certain embodiments, an increase in translation refers to an increase in the number of polypeptide (e.g. a protein) molecules that are made per copy of mRNA that encodes said polypeptide.

The term “target nucleic acid” refers to a nucleic acid molecule to which an antisense oligonucleotide is intended to hybridize.

The term “mRNA” refers to an RNA molecule that encodes a protein.

The term “pre-mRNA” refers to an RNA transcript that has not been fully processed into mRNA. A pre-RNA may include one or more introns.

The terms “targeting” and “targeted to” refer to the association of an antisense oligonucleotide to a particular target nucleic acid molecule or a particular region of a target nucleic acid molecule. An antisense oligonucleotide targets a target nucleic acid if it is sufficiently complementary to the target nucleic acid to allow hybridization under physiological conditions.

When in reference to nucleobases, the terms “nucleobase complementarity” and “complementarity” refer to a nucleobase that is capable of base pairing with another nucleobase. For example, in DNA, adenine (A) is complementary to thymine (T). For example, in RNA, adenine (A) is complementary to uracil (U). Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. In certain embodiments, a complementary nucleobase refers to a nucleobase of an antisense oligonucleotide that is capable of base pairing with a nucleobase of its target nucleic acid. For example, if a nucleobase at a certain position of an antisense oligonucleotide is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

When used in reference to nucleobases, the term “non-complementary” refers to a pair of nucleobases that do not form hydrogen bonds with one another.

When used in reference to oligomeric compounds (e.g., linked nucleosides, oligonucleotides, or nucleic acids), the term “complementary” refers to the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

The term “mismatch” refers to a nucleotide of a first polynucleotide that is not capable of pairing with a nucleotide at a corresponding position of a second polynucleotide, when the first and second polynucleotide are aligned.

The term “hybridization” refers to the pairing of complementary oligomeric compounds (e.g., an antisense oligonucleotide and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

The term “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

When used in reference to an oligonucleotide or portion thereof, the term “fully complementary” means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand.

The term “percent complementarity” refers to the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

The term “percent identity” refers to the number of nucleobases in a first nucleic acid that are the same type (independent of chemical modification) as nucleobases at corresponding positions in a second nucleic acid, divided by the total number of nucleobases in the first nucleic acid.

The term “modulation” refers to a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

The term “modification motif” refers to a pattern of chemical modifications in an oligomeric compound or a region thereof. Motifs may be defined by modifications at certain nucleosides and/or at certain linking groups of an oligomeric compound.

The term “nucleoside motif” refers to a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

The term “sugar motif” refers to a pattern of sugar modifications in an oligomeric compound or a region thereof.

The term “linkage motif” refers to a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

The term “nucleobase modification motif” refers to a pattern of modifications to nucleobases along an oligonucleotide. Unless otherwise indicated, a nucleobase modification motif is independent of the nucleobase sequence.

The term “sequence motif” refers to a pattern of nucleobases arranged along an oligonucleotide or portion thereof. Unless otherwise indicated, a sequence motif is independent of chemical modifications and thus may have any combination of chemical modifications, including no chemical modifications.

The term “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

The term “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

The term “the same type of modifications” refers to modifications that are the same as one another, including absence of modifications. Thus, for example, two unmodified DNA nucleoside have “the same type of modification,” even though the DNA nucleoside is unmodified. Such nucleosides having the same type modification may comprise different nucleobases.

The phrase “pharmaceutically acceptable carrier or diluent” refers to any substance suitable for use in administering to an animal. In certain embodiments, a pharmaceutically acceptable carrier or diluent is sterile saline. In certain embodiments, such sterile saline is pharmaceutical grade saline.

The terms “substituent” and “substituent group” refer to an atom or group that replaces the atom or group of a named parent compound. For example, a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present invention have substituents at one or at more than one position of the parent compound. Substituents may also be further substituted with other substituent groups and may be attached directly or via a linking group such as an alkyl or hydrocarbyl group to a parent compound.

When used in reference to a chemical functional group, the term “substituent” refers to an atom or group of atoms that differs from the atom or group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Raa), carboxyl (—C(O)O—Raa), aliphatic groups, alicyclic groups, alkoxy, substituted oxy (—O—Raa), aryl, aralkyl, heterocyclic radical, heteroaryl, heteroarylalkyl, amino (—N(Rbb)(Rcc)), imino (═NRbb), amido (—C(O)N(Rbb)(Rcc) or —N(Rbb)C(O)Raa), azido (—N₃), nitro (—NO₂), cyano (—CN), carbamido (—OC(O)N(Rbb)(Rcc) or —N(Rbb)C(O)ORaa), urcido (—N(Rbb)C(O)N(Rbb)(Rcc)), thiourcido (—N(Rbb)C(S)N(Rbb)—(Rcc)), guanidinyl (—N(Rbb)C(═NRbb)N(Rbb)(Rcc)), amidinyl (—C(═NRbb)N(Rbb)(Rcc) or —N(Rbb)C(═NRbbb)(Raa)), thiol (—SRbb), sulfinyl (—S(O)Rbb), sulfonyl (—S(O)₂Rbb) and sulfonamidyl (—S(O)₂N(Rbb)(Rcc) or —N(Rbb)S—(O)₂Rbb), wherein each Raa, Rbb and Rcc is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

The term “alkyl” refers to a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred.

The term “alkenyl” refers to a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

The term “alkynyl” refers to a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

The term “acyl” refers to a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

The term “alicyclic” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

The term “aliphatic” refers to a straight or branched hydrocarbon radical containing up to 24 carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines Aliphatic groups as used herein may optionally include further substituent groups.

The term “alkoxy” refers to a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

The term “aminoalkyl” refers to an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

The term “aralkyl” and “arylalkyl” mean an aromatic group that is covalently linked to a C1-C12 alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

The term “aryl” and “aromatic” mean a mono-or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

The term “halo” and “halogen,” mean an atom selected from fluorine, chlorine, bromine and iodine.

The term “heteroaryl,” and “heteroaromatic,” mean a radical comprising a mono-or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

The compounds described herein include variations in which one or more atoms are replaced with a non-radioactive isotope or radioactive isotope of the indicated element. For example, compounds herein that comprise hydrogen atoms encompass all possible deuterium substitutions for each of the 1H hydrogen atoms.

Isotopic substitutions encompassed by the compounds herein include but are not limited to e.g., 2H or 3H in place of 1H, 13C or 14C in place of 12C, 15N in place of 14N, 17O or 18O in place of 16O, and 33S, 34S, 35S, or 36S in place of 32S etc. In certain embodiments, non-radioactive isotopic substitutions may impart new properties on the oligomeric compound that are beneficial for use as a therapeutic or research tool. In certain embodiments, radioactive isotopic substitutions may make the compound suitable for research purposes such as imaging.

As used herein, the term “nanoparticle” refers to any particle having an average diameter of less than 500 nanometers (nm). In some embodiments, nanoparticles have an average diameter of less than 300 nm, less than 100 nm, less than 50 nm, less than 25 nm, less than 10 nm or less than 5 nm. In some embodiments, each nanoparticle has a diameter of less than 300 nm, less than 100 nm, less than 50 nm, less than 25 nm, less than 10 nm or less than 5 nm.

As used herein, the term “hypertrophy” refers to an increase in mass of an organ or structure independent of natural growth that does not involve tumor formation. Hypertrophy of an organ or tissue is due either to an increase in the mass of the individual cells (true hypertrophy), or to an increase in the number of cells making up the tissue (hyperplasia), or both. Certain organs, such as the heart, lose the ability to divide shortly after birth.

The term “cardiac hypertrophy” refers to an increase in mass of the heart, which, in adults, is characterized by an increase in myocyte cell size and contractile protein content without concomitant cell division. The character of the stress responsible for inciting the hypertrophy, (e.g., increased preload, increased afterload, loss of myocytes, as in myocardial infarction, or primary depression of contractility, etc.), appears to play a critical role in determining the nature of the response. The early stage of cardiac hypertrophy is usually characterized morphologically by increases in the size of mycrofibrils and mitochondria, as well as enlargement of mitochondria and nuclei. At this stage, while muscle cells are larger than normal, cellular organization is largely preserved. At a more advanced stage of cardiac hypertrophy, there are preferential increases in the size or number of specific organelles, such as mitochondria, and new contractile elements are added in localized areas of the cells, in an irregular manner. Cells subjected to long-standing hypertrophy show more obvious disruptions in cellular organization, including markedly enlarged nuclei with highly lobulated membranes, which displace adjacent myofibrils and cause breakdown of normal Z-band registration. The phrase “cardiac hypertrophy” is used to include all stages of the progression of this condition, characterized by various degrees of structural damage of the heart muscle, regardless of the underlying cardiac disorder. In certain aspects, the cardiac hypertrophy may result from a number of causes, including idiopathic, cardiotrophic, or myotrophic causes, ischemia, or ischemic insults, such as myocardial infarction. In certain aspects the cardiac hypertrophy is associated with a pathological condition selected from hypertension, aortic stenosis, myocardial infarction, cardiomyopathy (including hypertrophic cardiomyopathy), valvular regurgitation, cardiac shunt, and heart failure (including congestive heart failure).

The term “heart failure” refers to an abnormality of cardiac function where the heart does not pump blood at the rate needed for the requirements of metabolizing tissues. The heart failure can be caused by a number of factors, including ischemic, congenital, rheumatic, or idiopathic forms.

The term “congestive heart failure” refers to a progressive pathologic state where the heart is increasingly unable to supply adequate cardiac output (the volume of blood pumped by the heart over time) to deliver the oxygenated blood to peripheral tissues. As congestive heart failure progresses, structural and hemodynamic damages occur. While these damages have a variety of manifestations, one characteristic symptom is ventricular hypertrophy. Congestive heart failure is a common end result of a number of various cardiac disorders.

The term “myocardial infarction” refers to a condition resulting from atherosclerosis of the coronary arteries, often with superimposed coronary thrombosis. It may be divided into two major types: transmural infarcts, in which myocardial necrosis involves the full thickness of the ventricular wall, and subendocardial (nontransmural) infarcts, in which the necrosis involves the subendocardium, the intramural myocardium, or both, without extending all the way through the ventricular wall to the epicardium. Myocardial infarction is known to cause both a change in hemodynamic effects and an alteration in structure in the damaged and healthy zones of the heart. Thus, for example, myocardial infarction reduces the maximum cardiac output and the stroke volume of the heart. Also associated with myocardial infarction is a stimulation of the DNA synthesis occurring in the interstice as well as an increase in the formation of collagen in the areas of the heart not affected.

As used herein, the term “treating,” “treatment,” and the like relate to any treatment of a target disease or condition, including but not limited to prophylactic treatment and therapeutic treatment. “Treating” includes any effect, e.g., lessening, reducing, modulating, or eliminating, that results in the improvement of a target disease or condition, such as cardiac hypertrophy or cardiac fibrosis. Those in need of treatment include those already with target disease or condition, and those in whom the target disease or condition is to be prevented.

As used herein, the term “subject” refers to a mammal, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). A “subject in need thereof” refers to a subject who may have, is diagnosed with, is suspected of having, or requires prevention of a disease or condition.

An “effective amount” or a “therapeutically effective amount” is defined herein in relation to the treatment of a target disease or condition as an amount that when administered alone or in combination with another therapeutic agent to a cell, tissue, or subject is effective to decrease, reduce, inhibit, or otherwise abrogate the development of the target disease or condition. An “effective amount” further refers to that amount of the compound sufficient to result in healing, prevention, or amelioration of symptoms of the target disease or condition. The “effective amount” will vary depending the cause of the target disease or condition and the severity of the target disease or condition, as well as the age, weight, etc., of the subject to be treated. Additionally, the “effective amount” can vary depending upon the dosage form employed and the route of administration utilized. A physician or veterinarian having ordinary skill in the art can readily determine and prescribe the effective amount (e.g., ED50) of the active ingredients required. For example, the physician or veterinarian can start doses of the administered compounds at levels lower than that required in order to achieve the desired therapeutic effect and gradually increase the dosage until the desired effect is achieved.

The term “disease-related genes” refers to genes that encode proteins involved in the a disease process, including proteins that participate the disease process directly and proteins that modulate the disease process. Disease-related genes include pro-disease genes and anti-disease genes. Expression of a pro-disease gene facilitates the development of the disease, while expression of an anti-disease gene inhibits or slows down disease development.

The term “fibrosis-related genes” refers to genes that encode proteins involved in the fibrosis process, including proteins that participate the fibrosis process directly, such as extracellular matrix proteins, and proteins that modulate the fibrosis process, such as GATA 4 and eIF4G2 proteins. Fibrosis-related genes include pro-fibrosis genes and anti-fibrosis genes. Expression of a pro-fibrosis gene facilitates fibrosis, while expression of an anti-fibrosis gene inhibits fibrosis.

The term “cardiac fibrosis-related genes” refers to fibrosis-related genes that are involved in the fibrosis process in the heart. Example of cardiac fibrosis-related genes include, but not limited to, pro-fibrosis genes such as eukaryotic translation initiation factor 4gamma 2 (cIF4G2), glutamyl-prolyl-tRNA synthetase (EPRS) and mesenchyme Homeobox 1(MEOX1) and anti-fibrosis genes such as GATA binding protein 4 (GATA4), myocyte enhancer factor 2C (MEF2C), NK2 homeobox 5 (NKX2-5), T-box transcription factor 5(TBX5), hepatocyte nuclear factor 4 alpha (HNF4α), alpha crystalline B (CRYAB), transcription factor 21 (TCF21) and myosin binding protein C (MYBPC3).

The term “cardiac hypertrophy-related genes” refers to genes that are involved in the cardiac hypertrophy process. Example of cardiac hypertrophy-related genes include, but not limited to, GATA4.

### II. COMPOSITIONS FOR MODULATING TRANSLATION

Translation of a protein encoded by a messenger ribonucleic acid (mRNA) usually begins at the start codon of the main open reading frame (mORF) of the mRNA. Some mRNAs contain one or more upstream ORFs (uORFs) located in the 5′ untranslated region of mRNAs. uORFs have been established as a negative regulatory element to repress the translation of mORFs when their corresponding uORF is translated. Antisense oligonucleotide (ASO) technology provides an effective means for modulating the expression of specific mRNAs or proteins based on Watson-Crick base-pairing between an appropriately designed ASO and its target mRNA. ASO technology has been used most often to reduce the amount an mRNA via antisense induced RNase H cleavage or to alter splicing of a pre-mRNA transcript in a cell. In contrast, the present application provides antisense oligonucleotides (ASOs) and modified antisense oligonucleotides that are not designed to clicit cleavage. Specifically, the present disclosure provides antisense oligonucleotides and modified oligonucleotides that can selectively increase or decrease translation of a desired target protein in a cell by interrupting a double stranded region in an uORF of an mRNA (hereinafter referred to as Type I uotASO or ASO1), or forming an intermolecular double-stranded region that is downstream of, and adjacent to, a uORF start codon or a mORF start codon (hereinafter generally referred to as Type II ASO or ASO2).

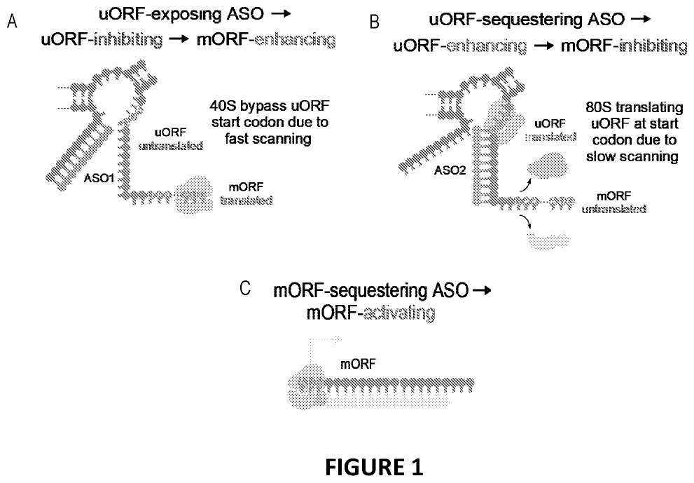

Although not wishing to be bound by theory, it is the inventors hypothesis that type I uotASOs (e.g., ASO1) disrupts the original double-stranded structure in the uORF by forming a double-stranded structure with the non-coding strand of the original double-stranded structure, thus inhibiting translation of the uORF, which in turn results in enhanced translation of the corresponding mORF (FIG. 1, Panel A). Although not wishing to be bound by theory, the suppressed uORF translation and enhanced mORF translation is believed to occur as a result of exposing the uORF region to facilitate fast scanning mediated by the 40S ribosomal subunit, thereby causing the 80S ribosomal subunit to enhance translational initiation translation at the mORF start codon and suppress translation initiation at the uORF start codon as illustrated in FIG. 1, Panel A.

In contrast, type II ASOs disrupts the original double-stranded structure in the uORF by forming a double-stranded structure with the coding strand of the original double-stranded structure, thus enhancing translation of the uORF, which in turn results in reduced translation of the corresponding mORF (FIG. 1, Panel B). Furthermore, type II ASOs may function in the absence of the original double-stranded structure in the uORF. In this case, type II ASOs may be designed to form a double-stranded structure with a target sequence downstream of, and adjacent to, the start codon of a uORF, forming a double-stranded structure with the target sequence downstream of the uORF start codon, thus enhancing translation of the uORF, which in turn results in reduced translation of the corresponding mORF. These uORF-targeting type II ASOs are referred to as “type II uotASOs”. In some embodiments, the target region includes regions that are two to eight nucleotides away from the adenine (A) of the uORF AUG start codon.

Although not wishing to be bound by theory, the enhanced mORF translation is believed occur as result of the type II uotASO forming an ASO/mRNA hybrid double-stranded structure with the uORF downstream of, and adjacent to, the uORF start codon. The hybrid ds RNA structure facilitates slow scanning mediated by the 40S ribosomal subunit, thereby causing the 80S ribosomal subunit to enhance translational initiation translation at the uORF start codon and suppress translation initiation at the mORF start codon as illustrated in FIG. 1, Panel B.

In addition, type II ASOs may be designed to form a double-stranded structure with a target sequence downstream of, and adjacent to, the start codon of a mORF, forming a double-stranded structure with the target sequence downstream of the mORF start codon, thus enhancing translation of the mORF. These mORF-targeting type II ASOs are referred to as “type II motASOs”. In some embodiments, the target region includes regions that are two to eight nucleotides away from the adenine (A) of the mORF AUG start codon.

Although not wishing to be bound by theory, the enhanced mORF translation by is believed occur as result of the type II motASO forming an ASO/mRNA hybrid double-stranded structure, which facilitates slow scanning mediated by the 40S ribosomal subunit, thereby causing the 80S ribosomal subunit to enhance translational initiation translation at the mORF start codon as illustrated in FIG. 1, Panel C.

One aspect of the present application is directed to antisense oligonucleotides (ASOs) that is capable of selectively increase or decrease translation of a disease-related gene. The design and use of these ASOs can be utilized for treatment or prevention of a disease or condition that is caused by, or related to, the disease-related gene.

In some embodiments, the ASO of the present application comprises a nucleotide capable of binding to a target region in an upstream open reading frame (uORF) of a mRNA of a target gene, wherein the target region forms a double-stranded stem structure with a region of the uORF that is downstream of, and adjacent to, the start codon of the uORF. The binding of the ASO to the target region disrupts the double-stranded stem structure of the uORF and enhances translation of the downstream mORF of the mRNA of the target gene.

In some embodiments, the target region has a length of between 5 to 30, 3 to 25, 3 to 20, 5 to 16, 5 to 12, 8 to 30, 8 to 25, 8 to 20, 8 to 16, 8 to 12, 12 to 30, 12 to 25, 12 to 20, 12 to 16, 16 to 30, 16 to 25, or 16 to 20 nucleotides. In some embodiments, the target gene is an anti-fibrosis gene. In some embodiments, the anti-fibrosis gene is selected from the group consisting of GATA4, MEF2C, NKX2-5, TBX5, DACHI, HNF4α, CRYAB, TCF21 and MYBPC3. In some embodiments, the target region comprises SEQ ID NO:59.

In some embodiments, the ASO comprises a sequence that is at least 50%, 60%, 70%, 80% or 90% complementary to the sequence of SEQ ID NO:59. In some embodiments, the ASO has a length between 8 to 50, 8 to 30, 8 to 25, 8 to 20, 8 to 16, 8 to 12, 12 to 50, 12 to 30, 12 to 25, 12 to 20, 12-16, 16 to 50, 16 to 30, 16 to 25, or 16 to 20 nucleotides. In some embodiments, the ASO comprises one or more modified nucleotides. In some embodiments, the ASO comprises the nucleotide sequence of SEQ ID NO:10.

In some embodiments, the ASO of the present application comprises a nucleotide capable of binding to a target region in an uORF of a mRNA of a target gene, wherein the target region is downstream of, and adjacent to, the start codon of the uORF. The binding of the ASO to the target region forms a double-stranded ASO/mRNA structure that enhances translation of the uORF of the mRNA and inhibits translation of the corresponding mORF of the mRNA of the target gene.

In some embodiments, the target region has a length of between 5 to 30, 3 to 25, 3 to 20, 5 to 16, 5 to 12, 8 to 30, 8 to 25, 8 to 20, 8 to 16, 8 to 12, 12 to 30, 12 to 25, 12 to 20, 12 to 16, 16 to 30, 16 to 25, or 16 to 20 nucleotides. In some embodiments, the target region comprises SEQ ID NO:3, 4 or 60.

In some embodiments, the target gene is GATA4, TBX5, NKX2-5, MEF2C, TBX20, or MYOCD. In some embodiments, the target gene is a pro-fibrosis gene. In some embodiments, the pro-fibrosis gene is selected from the group consisting of eIF4G2, EPRS and MEOX1. In some embodiments, the target region comprises SEQ ID NO:60.

In some embodiments, the ASO comprises a sequence that is at least 50%, 60%, 70%, 80% or 90% complementary to the sequence of SEQ ID NO:3, 4 or 60. In some embodiments, the ASO has a length between 8 to 50, 8 to 30, 8 to 25, 8 to 20, 8 to 16, 8 to 12, 12 to 50, 12 to 30, 12 to 25, 12 to 20, 12-16, 16 to 50, 16 to 30, 16 to 25, or 16 to 20 nucleotides. In some embodiments, the ASO comprises one or more modified nucleotides. In some embodiments, the ASO comprises the nucleotide sequence of SEQ ID NO:5, 7, 29, 36, 52, 53, 54, 55, 56, 57, 58 or 61. In some embodiments, the ASO comprises the nucleotide sequence of SEQ ID NO:61.

In some embodiments, the 3′ end of the ASO includes at least one nucleotide complementary to a nucleotide within the uORF start codon. In certain embodiments, the 3′ end of the ASO includes a cytosine, which is complementary to the guanine in the AUG start codon of the uORF.

In some embodiments, the ASO comprises a nucleotide capable of binding to a target region in an mORF of a mRNA of a target gene, wherein the target region is downstream of, and adjacent to, the start codon of the mORF. The binding of the ASO to the target region forms a double-stranded ASO/mRNA structure that enhances translation of the mORF of the mRNA of the target gene.

In some embodiments, the target region has a length of between 5 to 30, 3 to 25, 3 to 20, 5 to 16, 5 to 12, 8 to 30, 8 to 25, 8 to 20, 8 to 16, 8 to 12, 12 to 30, 12 to 25, 12 to 20, 12 to 16, 16 to 30, 16 to 25, or 16 to 20 nucleotides. In some embodiments, the target region comprises SEQ ID NO:44, 47, 62 or 63.

In some embodiments, the target gene is GATA4, cIF4G2, TBX5, NKX2-5, MEF2C, TBX20, or MYOCD. In some embodiments, the target gene is an anti-fibrosis gene. In some embodiments, the anti-fibrosis gene is selected from the group consisting of GATA4, MEF2C, NKX2-5, TBX5, DACHI, HNF4α, CRYAB, TCF21 and MYBPC3. In some embodiments, the target region comprises SEQ ID NO:44.

In some embodiments, the ASO comprises a sequence that is at least 50%, 60%, 70%, 80% or 90% complementary to the sequence of SEQ ID NO:44, 47, 62 or 63. In some embodiments, the ASO has a length between 8 to 50, 8 to 30, 8 to 25, 8 to 20, 8 to 16, 8 to 12, 12 to 50, 12 to 30, 12 to 25, 12 to 20, 12-16, 16 to 50, 16 to 30, 16 to 25, or 16 to 20 nucleotides. In some embodiments, the ASO comprises one or more modified nucleotides. In some embodiments, the ASO comprises the nucleotide sequence of SEQ ID NO:28, 30, 31, 32, 33, 34, 35, 37 or 38.

In some embodiments, the 3′ end of the ASO includes at least one nucleotide complementary to a nucleotide within the mORF start codon. In certain embodiments, the 3′ end of the ASO includes a cytosine, which is complementary to the guanine in the AUG start codon of the mORF.

### Modified Nucleosides

ASOs of the present application may comprise or consist of oligonucleotides comprising at least one modified nucleoside. Such modified nucleosides may comprise a modified sugar moiety, a modified nucleobase, or both. In some embodiments, the ASO comprises at least 5, at least 10, at least 15, at least 20, at least 25 or more modified nucleosides relative to the total number of nucleosides in the ASO. In some embodiments, the modified ASO includes a modified region of at least 5, at least 10, at least 15, at least 20, at least 25 or more contiguous modified nucleosides in the ASO. In some embodiments, each of the nucleosides in the ASO is modified.

In certain preferred embodiments, the one or more modified nucleotides include a 2′-O-methyl modified sugar moiety and/or a modified internucleoside linkage. In some embodiments, the modified internucleoside linkage is a phosphodiester internucleoside linkage or a phosphorothioate internucleoside linkage.

In some embodiments, the ASO of the present application comprises one or more sugar-modified nucleotides. In some embodiments, the ASO comprises the nucleotide sequence of any one of SEQ ID NOs: 3-6 with one or more modified sugar moieties and/or modified internucleoside linkages.

In certain particular embodiments, the ASO comprises the nucleotide sequence of AmoCmoGmoUmoAmoUmoUmoAmoAmoAmoUmoCmoCmoAmoGmoCm (SEQ ID NO: 7), where “m” indicates a 2′-O-methyl modification, and “o” indicates a phosphodiester or phosphorothioate internucleoside linkage. It should be noted that in any of the sequences disclosed in the present application, where the modifications “o” or “mo” are included, such modifications may be substituted with any nucleoside modifications described herein or they may contain no nucleoside modifications at all.

### Sugar Moieties

The ASOs of the present application may contain nucleosides with naturally occurring sugar moieties and/or nucleosides with modified sugar moieties. ASOs comprising nucleosides with modified sugar moieties may have desirable properties, such as enhanced nuclease stability or increased binding affinity with a target nucleic acid relative to ASOs comprising only nucleosides comprising naturally occurring sugar moieties. In some embodiments, the modified sugar moieties are substituted sugar moieties. In certain embodiments, the modified sugar moieties are bicyclic or tricyclic sugar moieties. In certain embodiments, the modified sugar moieties are sugar surrogates. Such sugar surrogates may include one or more substitutions corresponding to those of substituted sugar moieties.

In certain embodiments, the modified sugar moieties are substituted sugar moieties comprising one or more substituent, including but not limited to substituents at the 2′ and/or 5′ positions. Examples of sugar substituents suitable for the 2′-position, include, but are not limited to: 2′-F, 2′-OCH3 (“O-methyl”), and 2′-O(CH₂)₂OCH₃. In certain embodiments, sugar substituents at the 2′ position is selected from allyl, amino, azido, thio, O-allyl, O—C1-C10 alkyl, O—C1-C10 substituted alkyl; O—C1-C10 alkoxy; O—C1-C10 substituted alkoxy, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O—C1-C10 alkoxy; O—C1-C10 substituted alkoxy, SH, CN, OCN, CF₃, OCF₃, O-alkyl, S-alkyl, N(Rm)-alkyl; O-alkenyl, S-alkenyl, or N(Rm)-alkenyl; O-alkynyl, S-alkynyl, N(Rm)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rm)(Rn) or O—CH₂—C(═O)—N(Rm)(Rn), where each Rm and Rn is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rm)(Rn), O(CH₂)₂O(CH₂)₂N(CH₃)₂, and N-substituted acetamide (O—CH₂—C(═O)—N(Rm)(Rn) where each Rm and Rn is, independently, H, an amino protecting group or substituted or unsubstituted C1-C10 alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Ra)(Rb)]n—, —[C(Ra)(Rb)]n—O—, —C(RaRb)—N(R)—O— or, —C(RaRb)—O—N(R)—; 4′-CH₂-2′, 4′-(CH₂)2-2′, 4′-(CH₂)3-2′, 4′-(CH₂)—O-2′(LNA); 4′-(CH2)—S-2; 4′-(CH2) 2—O-2′(ENA); 4′-CH(CH3)—O-2′(cEt) and 4′-CH(CH₂OCH₃)—O-2′, and analogs thereof (see, e.g., U.S. Pat. No. 7,399,845, issued on Jul. 15, 2008); 4′-C (CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)-0-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, e.g., Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Ra)(Rb)]n—, —C(Ra)═C(Rb)—, —C(Ra)═N—, —C(═NRa)—, —C(═O)—, —C(═S)—, —O—, —Si(Ra)₂—, —S(═O)x—, and —N(Ra)—;
wherein:
x is 0, 1, or 2;
n is 1, 2, 3, or 4;
each Ra and Rb is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ1, NJ1J2, SJ1, N3, COOJ1, acyl (C(═O)—H), substituted acyl, CN, sulfonyl (S(═O)₂-J1), or sulfoxyl (S(═O)-J1); and each J1 and J2 is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH2—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂-N(R)-2′) BNA, (I) methyl carbocyclic (4′-CH₂—CH(CH₃)-2′) BNA, (J) propylene carbocyclic (4′-(CH₂)₃-2′) BNA, and (M) 4′-CH₂—O—CH₂-2′ as depicted below.

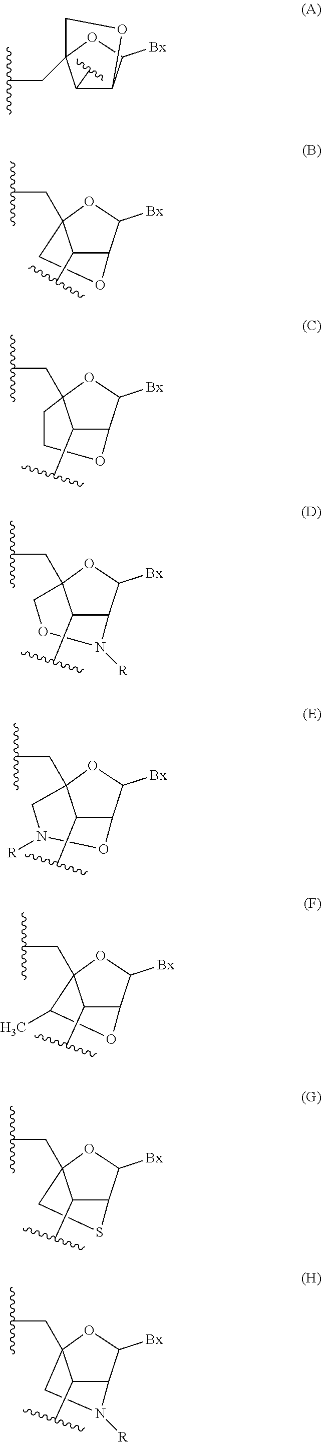

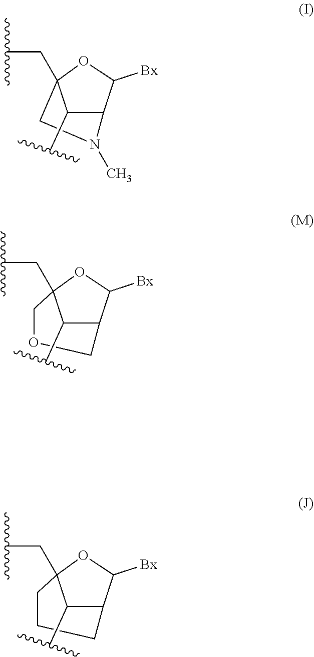

wherein Bx is a nucleobase moiety and R is, independently, H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfer, carbon or nitrogen atom. In certain such embodiments, such modified sugar moiety also comprises bridging and/or non-bridging substituents as described above. For example, certain sugar surrogates comprise a 4′-sulfer atom and a substitution at the 2′-position (see, e.g., published U.S. Patent Application US2005/0130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), 34damanti nucleic acid (MNA) (see Leumann, C J. Bioorg. & Med. Chem. (2002) 10:841-854), fluoro HNA (F-HNA), and those compounds having Formula VII:

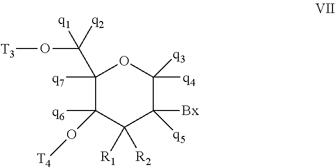

wherein independently for each of said at least one tetrahydropyran nucleoside analog of Formula VII:
Bx is a nucleobase moiety;
T3 and T4 are each, independently, an internucleoside linking group linking the tetrahydropyran nucleoside analog to the antisense oligonucleotide or one of T3 and T4 is an internucleoside linking group linking the tetrahydropyran nucleoside analog to the antisense oligonucleotide and the other of T3 and T4 is H, a hydroxyl protecting group, a linked conjugate group, or a 5′ or 3′-terminal group;
q1, q2, q3, q4, q5, q6 and q7 are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R1 and R2 is independently selected from among: hydrogen, halogen, substituted or unsubstituted alkoxy, NJ1J2, SJ1, N3, OC(═X)J1, OC(═X)NJ1J2, NJ3C(═X)NJ1J2, and CN, wherein X is O, S or NJ1, and each J1, J2, and J3 is, independently, H or C₁-C₆ alkyl.

In certain embodiments, the modified THP nucleosides of Formula VII are provided wherein q1, q2, q3, q4, q5, q6 and q7 are each H. In certain embodiments, at least one of q1, q2, q3, q4, q5, q6 and q7 is other than H. In certain embodiments, at least one of q1, q2, q3, q4, q5, q6 and q7 is methyl. In certain embodiments, THP nucleosides of Formula VII are provided wherein one of R1 and R2 is F. In certain embodiments, R1 is fluoro and R2 is H, R1 is methoxy and R2 is H, and R1 is methoxyethoxy and R2 is H.

Many other bicyclic and tricyclic sugar and sugar surrogate ring systems are known in the art that can be used to modify nucleosides (see, e.g., review article: Leumann, J C, Bioorganic & Medicinal Chemistry, 2002, 10, 841-854).

In certain embodiments, sugar surrogates comprise rings having more than 5 atoms and more than one heteroatom. For example, nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

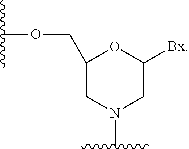

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157Published on Aug. 21, 2008 for other disclosed 5′,2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH2—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

### Modified Nucleobases

In certain embodiments, nucleosides of the present invention comprise one or more unmodified nucleobases. In certain embodiments, nucleosides of the present invention comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido [5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

### Internucleoside Linkages

In certain embodiments, nucleosides may be linked together using any internucleoside linkage to form oligonucleotides. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH2—N(CH₃)—O—CH2—), thiodiester (—O—C(O)—S—), thionocarbamate (—O—C(O)(NH)—S—); siloxane (—O—Si(H)₂—O—); and N,N′-dimethylhydrazine (—CH2—N(CH₃)—N(CH₃)—). Modified linkages, compared to natural phosphodiester linkages, can be used to alter, typically increase, nuclease resistance of the oligonucleotide. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense oligonucleotides provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### Motifs

In some embodiments, the ASO of the present application comprises a modified oligonucleotide. In some embodiments, the modified oligonucleotide comprises one or more modified sugars. In some embodiments, the modified oligonucleotide comprises one or more modified nucleobases. In some embodiments, the modified oligonucleotide comprises one or more modified internucleoside linkages. In some embodiments, the modifications (sugar modifications, nucleobase modifications, and/or linkage modifications) define a pattern or motif. In some embodiments, the patterns of chemical modifications of sugar moieties, internucleoside linkages, and nucleobases are each independent of one another. Thus, a modified oligonucleotide may be described by its sugar modification motif, internucleoside linkage motif and/or nucleobase modification motif (as used herein, nucleobase modification motif describes the chemical modifications to the nucleobases independent of the sequence of nucleobases).

In certain embodiments, every sugar moiety of the modified oligonucleotides of the present invention is modified. In certain embodiments, modified oligonucleotides include one or more unmodified sugar moiety.

### Overall Lengths

In certain embodiments, the present invention provides modified oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that XSY. For example, in certain embodiments, the invention provides modified oligonucleotides which comprise oligonucleotides consisting of 5 to 6, 5 to 7, 5 to 8, 5 to 9, 5 to 5 to 10, 5 to 11, 5 to 12, 5 to 13, 5 to 14, 5 to 15, 5 to 16, 5 to 17, 5 to 18, 5 to 19, 5 to 20, 6 to 7 6 to 8, 6 to 9, 6 to 10, 6 to 11, 6 to 12, 6 to 13, 6 to 14, 6 to 15, 6 to 16, 6 to 17, 6 to 18, 6 to 19, 6 to 20, 7 to 8, 7 to 9, 7 to 10, 7 to 11, 7 to 12, 7 to 13, 7 to 14, 7 to 15, 7 to 16, 7 to 17, 7 to 18, 7 to 19, 7 to 20, 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a modified oligonucleotide has any of the above lengths.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range.

In certain embodiments, oligonucleotides of the present application are characterized by their modification motif and overall length. In certain embodiments, such parameters are each independent of one another.

### Oligomeric Compounds

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

In certain embodiments, antisense oligonucleotides are provided wherein the 5′-terminal group comprises a 5′-terminal stabilized phosphate. A “5′-terminal stabilized phosphate” is a 5′-terminal phosphate group having one or more modifications that increase nuclease stability relative to a 5′-phosphate.

In certain embodiments, antisense oligonucleotides are provided wherein the 5′-terminal group has Formula IIe:

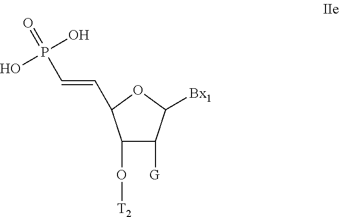

wherein:
Bx is uracil, thymine, cytosine, 5-methyl cytosine, adenine or guanine;
T2 is a phosphorothioate internucleoside linking group linking the compound of Formula lie to the oligomeric compound; and
G is halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂ or OCH₂—N(H)—C(═NH)NH₂.

In certain embodiments, antisense oligonucleotides are provided wherein said 5′-terminal compound has Formula IIe wherein G is F, OCH₃ or O(CH₂)₂—OCH₃.

In certain embodiments, the 5′-terminal group is a 5′-terminal stabilized phosphate comprising a vinyl phosphonate represented by Formula IIe above.

### Conjugate Groups

In certain embodiments, the ASO of the present application comprises an antisense oligonucleotide modified by covalent attachment of one or more conjugate groups (also referred to as “conjugate partner”). In general, conjugate groups modify one or more properties of the attached oligonucleotide including but not limited to pharmacodynamics, pharmacokinetics, stability, binding, absorption, cellular distribution, cellular uptake, charge and clearance. As used herein, “conjugate group” means a radical group comprising a group of atoms that are attached to an oligonucleotide or oligomeric compound. In general, conjugate groups modify one or more properties of the compound to which they are attached, including but not limited to, pharmacodynamics, pharmacokinetics, binding, absorption, cellular distribution, cellular uptake, charge and/or clearance properties. Conjugate groups are routinely used in the chemical arts and can include a conjugate linker that covalently links the conjugate group to an oligonucleotide or oligomeric compound. In certain embodiments, conjugate groups include a cleavable moiety that covalently links the conjugate group to an oligonucleotide or oligomeric compound. In certain embodiments, conjugate groups include a conjugate linker and a cleavable moiety to covalently link the conjugate group to an oligonucleotide or oligomeric compound. In certain embodiments, a conjugate group has the general formula:

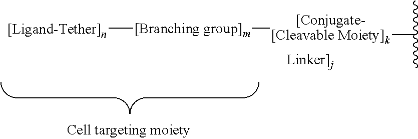

wherein n is from 1 to about 3, m is 0 when n is 1 or m is 1 when n is 2 or 3, j is 1 or 0, k is 1 or 0 and the sum of j and k is at least one.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

Conjugate groups are shown herein as radicals, providing a bond for forming covalent attachment to an oligomeric compound such as an oligonucleotide. In certain embodiments, the point of attachment on the oligomeric compound is at the 3′-terminal nucleoside or modified nucleoside. In certain embodiments, the point of attachment on the oligomeric compound is the 3′-oxygen atom of the 3′-hydroxyl group of the 3′ terminal nucleoside or modified nucleoside. In certain embodiments, the point of attachment on the oligomeric compound is at the 5′-terminal nucleoside or modified nucleoside. In certain embodiments the point of attachment on the oligomeric compound is the 5′-oxygen atom of the 5′-hydroxyl group of the 5′-terminal nucleoside or modified nucleoside. In certain embodiments, the point of attachment on the oligomeric compound is at any reactive site on a nucleoside, a modified nucleoside or an internucleoside linkage.

As used herein, “cleavable moiety” and “cleavable bond” mean a cleavable bond or group of atoms that is capable of being split or cleaved under certain physiological conditions. In certain embodiments, a cleavable moiety is a cleavable bond. In certain embodiments, a cleavable moiety comprises a cleavable bond. In certain embodiments, a cleavable moiety is a group of atoms. In certain embodiments, a cleavable moiety is selectively cleaved inside a cell or sub-cellular compartment, such as a lysosome. In certain embodiments, a cleavable moiety is selectively cleaved by endogenous enzymes, such as nucleases. In certain embodiments, a cleavable moicty comprises a group of atoms having one, two, three, four, or more than four cleavable bonds.

In certain embodiments, conjugate groups comprise a cleavable moiety. In certain such embodiments, the cleavable moiety covalently attaches the oligomeric compound to the conjugate linker. In certain such embodiments, the cleavable moiety covalently attaches the oligomeric compound to the cell-targeting moiety.

In certain embodiments, a cleavable bond is selected from among an amide, a polyamide, an ester, an ether, one or both esters of a phosphodiester, a phosphate ester, a carbamate, a di-sulfide, or a peptide. In certain embodiments, a cleavable bond is one of the esters of a phosphodiester. In certain embodiments, a cleavable bond is one or both esters of a phosphodiester. In certain embodiments, the cleavable moiety is a phosphodiester linkage between an oligomeric compound and the remainder of the conjugate group. In certain embodiments, the cleavable moiety comprises a phosphodiester linkage that is located between an oligomeric compound and the remainder of the conjugate group. In certain embodiments, the cleavable moiety comprises a phosphate or phosphodiester. In certain embodiments, the cleavable moiety is attached to the conjugate linker by either a phosphodiester or a phosphorothioate linkage. In certain embodiments, the cleavable moicty is attached to the conjugate linker by a phosphodiester linkage. In certain embodiments, the conjugate group does not include a cleavable moiety.

In certain embodiments, the cleavable moiety is a cleavable nucleoside or a modified nucleoside. In certain embodiments, the nucleoside or modified nucleoside comprises an optionally protected heterocyclic base selected from a purine, substituted purine, pyrimidine or substituted pyrimidine. In certain embodiments, the cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine.

In certain embodiments, the cleavable moiety is 2′-deoxy nucleoside that is attached to either the 3′ or 5′-terminal nucleoside of an oligomeric compound by a phosphodiester linkage and covalently attached to the remainder of the conjugate group by a phosphodiester or phosphorothioate linkage. In certain embodiments, the cleavable moiety is 2′-deoxy adenosine that is attached to either the 3′ or 5′-terminal nucleoside of an oligomeric compound by a phosphodiester linkage and covalently attached to the remainder of the conjugate group by a phosphodiester or phosphorothioate linkage. In certain embodiments, the cleavable moiety is 2′-deoxy adenosine that is attached to the 3′-oxygen atom of the 3′-hydroxyl group of the 3′-terminal nucleoside or modified nucleoside by a phosphodiester linkage. In certain embodiments, the cleavable moiety is 2′-deoxy adenosine that is attached to the 5′-oxygen atom of the 5′-hydroxyl group of the 5′-terminal nucleoside or modified nucleoside by a phosphodiester linkage. In certain embodiments, the cleavable moicty is attached to a 2′-position of a nucleoside or modified nucleoside of an oligomeric compound.

As used herein, “conjugate linker” in the context of a conjugate group means a portion of a conjugate group comprising any atom or group of atoms that covalently link the cell-targeting moicty to the oligomeric compound either directly or through the cleavable moicty. In certain embodiments, the conjugate linker comprises groups selected from alkyl, amino, oxo, amide, disulfide, polyethylene glycol, ether, thiocther (—S—) and hydroxylamino (—O—N(H)—). In certain embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and other groups. In certain embodiments, the conjugate linker comprises at least one phosphorus linking group. In certain embodiments, the conjugate linker comprises at least one phosphodiester group. In certain embodiments, the conjugate linker includes at least one neutral linking group.

In certain embodiments, the conjugate linker is covalently attached to the oligomeric compound. In certain embodiments, the conjugate linker is covalently attached to the oligomeric compound and the branching group. In certain embodiments, the conjugate linker is covalently attached to the oligomeric compound and a tethered ligand. In certain embodiments, the conjugate linker is covalently attached to the cleavable moiety. In certain embodiments, the conjugate linker is covalently attached to the cleavable moiety and the branching group. In certain embodiments, the conjugate linker is covalently attached to the cleavable moiety and a tethered ligand. In certain embodiments, the conjugate linker includes one or more cleavable bonds. In certain embodiments, the conjugate group does not include a conjugate linker.

As used herein, “branching group” means a group of atoms having at least 3 positions that are capable of forming covalent linkages to two or more tether-ligands and the remainder of the conjugate group. In general, a branching group provides a plurality of reactive sites for connecting tethered ligands to the oligomeric compound through the conjugate linker and/or the cleavable moiety. In certain embodiments, the branching group comprises groups selected from alkyl, amino, oxo, amide, disulfide, polyethylene glycol, ether, thiocther and hydroxylamino groups. In certain embodiments, the branching group comprises a branched aliphatic group comprising groups selected from alkyl, amino, oxo, amide, disulfide, polyethylene glycol, ether, thiocther and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, the branching group is covalently attached to the conjugate linker. In certain embodiments, the branching group is covalently attached to the cleavable moiety. In certain embodiments, the branching group is covalently attached to the conjugate linker and each of the tethered ligands. In certain embodiments, the branching group comprises one or more cleavable bond. In certain embodiments, the conjugate group does not include a branching group.

In certain embodiments, conjugate groups as provided herein include a cell-targeting moiety that has at least one tethered ligand. In certain embodiments, the cell-targeting moiety comprises two tethered ligands covalently attached to a branching group. In certain embodiments, the cell-targeting moiety comprises three tethered ligands covalently attached to a branching group.

As used herein, “tether” means a group of atoms that connect a ligand to the remainder of the conjugate group. In certain embodiments, each tether is a linear aliphatic group comprising one or more groups selected from alkyl, substituted alkyl, ether, thioether, disulfide, amino, oxo, amide, phosphodiester and polyethylene glycol groups in any combination. In certain embodiments, each tether is a linear aliphatic group comprising one or more groups selected from alkyl, ether, thioether, disulfide, amino, oxo, amide and polyethylene glycol groups in any combination. In certain embodiments, each tether is a linear aliphatic group comprising one or more groups selected from alkyl, substituted alkyl, phosphodiester, ether and amino, oxo, amide groups in any combination. In certain embodiments, each tether is a linear aliphatic group comprising one or more groups selected from alkyl, ether and amino, oxo, amide groups in any combination. In certain embodiments, each tether is a linear aliphatic group comprising one or more groups selected from alkyl, amino and oxo groups in any combination. In certain embodiments, each tether is a linear aliphatic group comprising one or more groups selected from alkyl and oxo groups in any combination. In certain embodiments, each tether is a linear aliphatic group comprising one or more groups selected from alkyl and phosphodiester in any combination. In certain embodiments, each tether comprises at least one phosphorus linking group or neutral linking group.

In certain embodiments, tethers include one or more cleavable bond. In certain embodiments, each tethered ligand is attached to a branching group. In certain embodiments, each tethered ligand is attached to a branching group through an amide group. In certain embodiments, each tethered ligand is attached to a branching group through an ether group. In certain embodiments, each tethered ligand is attached to a branching group through a phosphorus linking group or neutral linking group. In certain embodiments, each tethered ligand is attached to a branching group through a phosphodiester group. In certain embodiments, each tether is attached to a ligand through either an amide or an ether group. In certain embodiments, each tether is attached to a ligand through an ether group.

In certain embodiments, each tether comprises from about 8 to about 20 atoms in chain length between the ligand and the branching group. In certain embodiments, each tether comprises from about 10 to about 18 atoms in chain length between the ligand and the branching group. In certain embodiments, each tether comprises about 13 atoms in chain length.

In certain embodiments, the present disclosure provides ligands wherein each ligand is covalently attached to the remainder of the conjugate group through a tether. In certain embodiments, each ligand is selected to have an affinity for at least one type of receptor on a target cell. In certain embodiments, ligands are selected that have an affinity for at least one type of receptor on the surface of a mammalian liver cell. In certain embodiments, ligands are selected that have an affinity for the hepatic asialoglycoprotein receptor (ASGP-R). In certain embodiments, each ligand is a carbohydrate. In certain embodiments, each ligand is, independently selected from galactose, N-acetyl galactoscamine, mannose, glucose, glucosamone and fucose. In certain embodiments, each ligand is N-acetyl galactoseamine (GalNAc). In certain embodiments, the targeting moicty comprises 1 to 3 ligands. In certain embodiments, the targeting moiety comprises 3 ligands. In certain embodiments, the targeting moiety comprises 2 ligands. In certain embodiments, the targeting moiety comprises 1 ligand. In certain embodiments, the targeting moiety comprises 3 N-acetyl galactoseamine ligands. In certain embodiments, the targeting moicty comprises 2 N-acetyl galactoseamine ligands. In certain embodiments, the targeting moicty comprises 1 N-acetyl galactoseamine ligand.

In certain embodiments, each ligand is a carbohydrate, carbohydrate derivative, modified carbohydrate, multivalent carbohydrate cluster, polysaccharide, modified polysaccharide, or polysaccharide derivative. In certain embodiments, each ligand is an amino sugar or a thio sugar. For example, amino sugars may be selected from any number of compounds known in the art, for example glucosamine, sialic acid, α-D-galactosamine, N-Acetylgalactosamine, 2-acetamido-2-deoxy-D-galactopyranose (GalNAc), 2-Amino-3-O—[®-1-carboxyethyl]-2-deoxy-β-D-glucopyranose (β-muramic acid), 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose and N-sulfo-D-glucosamine, and N-Glycoloyl-α-neuraminic acid. For example, thio sugars may be selected from the group consisting of 5-Thio-β-D-glucopyranose, Methyl 2,3,4-tri-O-acetyl-1-thio-6-O-trityl-α-D-glucopyranoside, 4-Thio-β-D-galactopyranose, and ethyl 3,4,6,7-tetra-O-acetyl-2-deoxy-1,5-dithio-α-D-gluco-heptopyranoside.

In certain embodiments, conjugate groups as provided herein comprise a carbohydrate cluster. As used herein, “carbohydrate cluster” means a portion of a conjugate group wherein two or more carbohydrate residues are attached to a branching group through tether groups. (see, e.g., Maier et al., “Synthesis of Antisense Oligonucleotides Conjugated to a Multivalent Carbohydrate Cluster for Cellular Targeting,” Bioconjugate Chemistry, 2003, (14): 18-29, which is incorporated herein by reference in its entirety, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808, for examples of carbohydrate conjugate clusters).

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative.

In certain embodiments, conjugate groups are provided wherein the cell-targeting moiety has the formula:

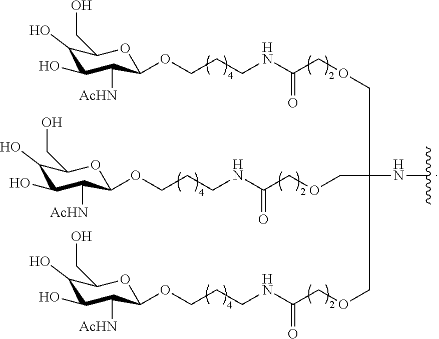

In certain embodiments, conjugate groups are provided wherein the cell-targeting moiety has the formula:

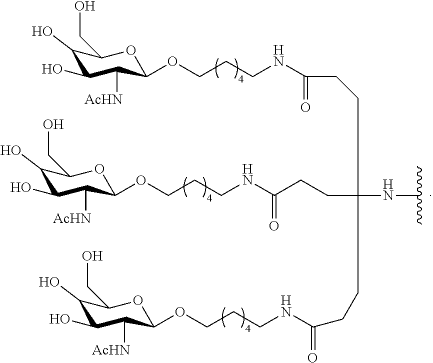

In certain embodiments, conjugate groups are provided wherein the cell-targeting moicty has the formula:

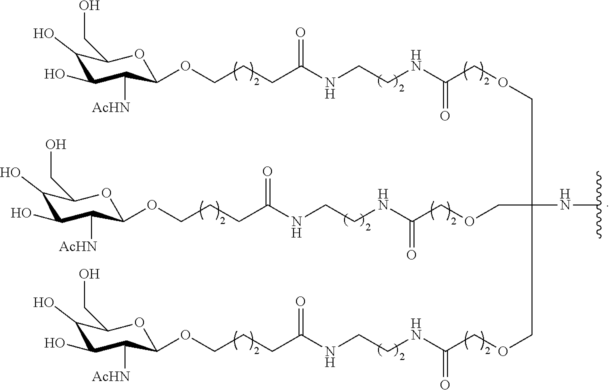

In certain embodiments, conjugate groups have the formula:

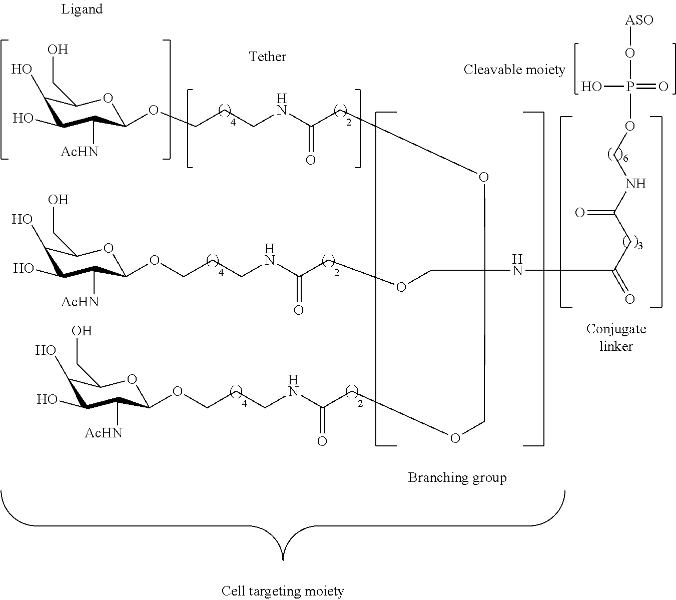

Representative United States patents, United States patent application publications, and international patent application publications that teach the preparation of certain of the above noted conjugate groups, conjugated oligomeric compounds such as ASOs comprising a conjugate group, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, each of which is incorporated by reference herein in its entirety.

Representative publications that teach the preparation of certain of the above noted conjugate groups, conjugated oligomeric compounds such as ASOs comprising a conjugate group, tethers, conjugate linkers, branching groups, ligands, cleavable moieties as well as other modifications include without limitation, BIESSEN et al., “The Cholesterol Derivative of a Triantennary Galactoside with High Affinity for the Hepatic Asialoglycoprotein Receptor: a Potent Cholesterol Lowering Agent” J. Med. Chem. (1995) 38:1846-1852, BIESSEN et al., “Synthesis of Cluster Galactosides with High Affinity for the Hepatic Asialoglycoprotein Receptor” J. Med. Chem. (1995) 38:1538-1546, LEE et al., “New and more efficient multivalent 51 daman-ligands for asialoglycoprotein receptor of mammalian hepatocytes” Bioorganic & Medicinal Chemistry (2011) 19:2494-2500, RENSEN et al., “Determination of the Upper Size Limit for Uptake and Processing of Ligands by the Asialoglycoprotein Receptor on Hepatocytes in Vitro and in Vivo” J. Biol. Chem. (2001) 276(40):37577-37584, RENSEN et al., “Design and Synthesis of Novel N-Acetylgalactosamine-Terminated Glycolipids for Targeting of Lipoproteins to the Hepatic Asialoglycoprotein Receptor” J. Med. Chem. (2004) 47:5798-5808, SLIEDREGT et al., “Design and Synthesis of Novel Amphiphilic Dendritic Galactosides for Selective Targeting of Liposomes to the Hepatic Asialoglycoprotein Receptor” J. Med. Chem. (1999) 42:609-618, and Valentijn et al., “Solid-phase synthesis of lysine-based cluster galactosides with high affinity for the Asialoglycoprotein Receptor” Tetrahedron, 1997, 53(2), 759-770, each of which is incorporated by reference herein in its entirety.

In certain embodiments, conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, 51 damantine, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thiocther, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Bchmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or 52damantine acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Some nonlimiting examples of conjugate linkers include pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′ end of an oligomeric compound, but before one or more terminal group nucleosides. In certain embodiments, conjugate groups are placed within a terminal group.

### III. PHARMACEUTICAL COMPOSITIONS FOR TREATING DISEASE

Another aspect of the present application relates to a pharmaceutical composition comprising one or more ASOs of the present application and a pharmaceutically acceptable carrier.

In some embodiments, the pharmaceutical composition comprises one or more carriers suitable for delivering the therapeutic agents to heart tissues. Exemplary carriers for delivery include nanoparticles, lipids, liposomes, micelles, polymers, polymeric micelles, emulsions, polyelectrolyte complexes, hydrogels, microcapsules, viruses, virus-like particle (VLPs), peptides, antibodies, aptamers, small molecule chemicals, exosomes, combinations thereof, and pegylated derivatives thereof. In a particular embodiment, the pharmaceutical composition comprises a nanoparticle formulation comprising an ASO in accordance with the present application.

In certain particular embodiments, the above-described carriers, including nanoparticles, may be linked to the heart tissue-specific targeting peptides or antibodies to facilitate carrier-mediated delivery of the active agents described herein to heart tissues. For example, in certain embodiments, pharmaceutical compositions include nanoparticles or liposomes covalently or non-covalently coated with a heart tissue-specific targeting peptide or antibody.

Exemplary nanoparticles include paramagnetic nanoparticles, superparamagnetic nanoparticles, metal nanoparticles, polymeric nanoparticles, nanoworms, nanoemulsions, nanogels, fullerene-like materials, inorganic nanotubes, dendrimers (such as with covalently attached metal chelates), nanocapsules, nanospheres, nanofibers, nanohoms, nano-onions, nanorods, nanoropes and quantum dots. A nanoparticle can produce a detectable signal, for example, through absorption and/or emission of photons (including radio frequency and visible photons) and plasmon resonance. Nanoparticles can be biodegradable or non-biodegradable.

In certain embodiments, the nanoparticle is a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, prascodymium, neodymium, promethium, samarium, curopium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199.

In other embodiments, a polymeric nanoparticle is made from a synthetic biodegradable polymer, a natural biodegradable polymer or a combination thereof. Synthetic biodegradable polymers can include, polyesters, such as poly(lactic-co-glycolic acid) (PLGA) and polycaprolactone; polyorthoesters, polyanhydrides, polydioxanones, poly-alkyl-cyano-acrylates (PAC), polyoxalates, polyiminocarbonates, polyurethanes, polyphosphazenes, or a combination thereof. Natural biodegradable polymers can include starch, hyaluronic acid, heparin, gelatin, albumin, chitosan, dextran, or a combination thereof.

In some embodiments, the pharmaceutical composition comprises a delivery carrier, such as a nanoparticle or liposome encapsulating a pharmaceutically effective amount of the antisense oligonucleotide. In some embodiments, the pharmaceutically effective amount of an ASO is from about 0.001 μg/mL to about 10 μg/mL (w/v) of the pharmaceutically acceptable carrier. In some embodiments, the pharmaceutically effective amount of ASO is from about 0.1 μg/mL to about 1 μg/mL (w/v) of the pharmaceutically acceptable carrier.

In some embodiments, the pharmaceutical composition comprises an ASO of the present application and a lipid moiety. Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono-or poly-cationic lipids are formed without the presence of a neutral lipid. In certain embodiments, the lipid moiety is selected to increase distribution of a pharmaceutical agent to heart tissue. In certain embodiments, the lipid moiety is selected to increase distribution of the pharmaceutical agent to heart muscle.

In certain embodiments, pharmaceutical compositions provided herein include one or more ASOs and one or more excipients. Exemplary excipients include water, salt solutions, alcohol, polyethylene glycols, gelatin, lactose, amylase, magnesium stearate, talc, silicic acid, viscous paraffin, hydroxymethylcellulose, polyvinylpyrrolidone and combinations thereof. In certain embodiments, the pharmaceutical compositions including one or more hydrophobic compounds, including organic solvents, such as dimethylsulfoxide.

In certain embodiments, the pharmaceutical composition provided herein comprises a co-solvent system. Co-solvent systems may include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied. For example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In some embodiments, the pharmaceutical composition comprises a sterile saline solution and one or more ASOs. In certain embodiments, the pharmaceutical composition consists of a sterile saline solution and one or more ASOs. In certain embodiments, the sterile saline is pharmaceutical grade saline. In certain embodiments, the pharmaceutical composition comprises one or more ASOs and sterile water. In certain embodiments, a pharmaceutical composition consists of one or more ASOs and sterile water. In certain embodiments, the sterile saline is pharmaceutical grade water. In certain embodiments, the pharmaceutical composition comprises one or more ASOs and phosphate-buffered saline (PBS). In certain embodiments, a pharmaceutical composition consists of one or more ASOs and sterile phosphate-buffered saline (PBS). In certain embodiments, the sterile saline is pharmaceutical grade PBS.

In certain embodiments, ASOs are admixed with pharmaceutically acceptable active and/or inert substances for the preparation of pharmaceutical compositions or formulations. Compositions and methods for the formulation of pharmaceutical compositions can depend on a number of criteria, including, but not limited to, route of administration, extent of disease, and/or dose to be administered.

Pharmaceutical compositions comprising ASOs may include any pharmaceutically acceptable salts, esters, or salts of such esters. In certain embodiments, pharmaceutical compositions comprising ASOs comprise one or more oligonucleotides, which, upon administration to an animal, such as a human, is capable of providing (directly or indirectly) the biologically active metabolite or residue thereof. Accordingly, for example, the disclosure is also drawn to pharmaceutically acceptable salts of ASOs, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active compound.

The pharmaceutical composition of the present application is formulated in accordance with the particular route of administration. Routes of administration for the therapeutic agents of the present application include oral and parenteral administration, i.e., injection, infusion, or implantation or by some other route other than the alimentary canal. Specific modes of administration include injections, such as intravenous, intramyocardial, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

In certain preferred embodiments, the pharmaceutical composition is formulated for administration by intravenous or intramyocardial injection. In certain embodiments, the pharmaceutical composition is formulated in aqueous solution, such as water or physiologically compatible buffers such as Hanks's solution, Ringer's solution, or physiological saline buffer. In certain embodiments, other ingredients are included (e.g., ingredients that aid in solubility or that serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Some pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### IV. METHODS FOR MODULATING GENE EXPRESSION

Another aspect of the present application relates to a method for modulating expression of a target gene in a subject. The method comprises the step of administering to the subject an effective amount of one or more ASOs of the present application.

Another aspect of the present application relates a method for treating a condition relating to the expression of a target gene in a subject. The method includes the step of administering to the subject an effective amount of an ASO of the present application.

In some embodiments, the condition is selected from the group consisting of cardiac hypertrophy, fibrosis, myocardial infarction, heart failure, hypertension, hyperlipidemia, thrombosis, cancer, and infectious diseases.

In some embodiments, the condition is selected from the group consisting of diseases or conditions caused by insufficiency of a functional protein encoded by a target gene and the ASO comprises a nucleotide capable of binding to a target region in an upstream open reading frame (uORF) of a mRNA of a target gene, wherein the target region forms a double-stranded stem structure with a region of the uORF that is downstream of, and adjacent to, the start codon of the uORF. The binding of the ASO to the target region disrupts the double-stranded stem structure of the uORF and enhances translation of the downstream mORF of the mRNA of the target gene. (Type I uotASO)

In some embodiments, the condition is selected from the group consisting of diseases or conditions caused by over-expression of a functional protein encoded by a target gene and the ASO comprises a nucleotide capable of binding to a target region in an uORF of a mRNA of the target gene, wherein the target region is downstream of, and adjacent to, the start codon of the uORF. The binding of the ASO to the target region forms a double-stranded ASO/mRNA structure that enhances translation of the uORF of the mRNA and inhibits translation of the corresponding mORF of the mRNA of the target gene. (Type II uotASO)

In some embodiments, the condition is selected from the group consisting of diseases or conditions caused by insufficiency of a functional protein encoded by a target gene and the ASO comprises a nucleotide capable of binding to a target region in an mORF of a mRNA of the target gene, wherein the target region is downstream of, and adjacent to, the start codon of the mORF. The binding of the ASO to the target region forms a double-stranded ASO/mRNA structure that enhances translation of the mORF of the mRNA of the target gene. (Type II motASO)

In some embodiments, the ASO is formulated in a nanoparticle formulation.

In some embodiments, the ASO is administered intravenously or intramyocardially.

In some embodiments, the ASO dosage may be expressed as the amount of the compound that results in amelioration of symptoms or a prolongation of survival in a patient. Toxicity and therapeutic efficacy of the ASO can be determined by standard pharmaceutical procedures in cell cultures or experimental animals (e.g., for determining the LD50—the dose lethal to 50% of the population) and the ED50 (the dose therapeutically effective in 50% of the population). The dose ratio between toxic and therapeutic effects is the therapeutic index, which can be expressed as the ratio between LD50 and ED50. Compounds exhibiting high therapeutic indices are preferred. The data obtained from cell culture assays and animal studies can be used in formulating a range of dosages or amounts for use in mammals (e.g., humans). The dosage or amount of an ASO preferably lies within a range of circulating concentrations that include the ED50 with little or no toxicity. The dosage or amount may vary within this range depending upon the dosage form employed and the route of administration utilized. The exact formulation, route of administration and dosage can be chosen by the individual physician in view of the patient's condition. Dosage amount and interval may be adjusted individually to provide plasma levels of the active moiety which are sufficient to maintain the desired effects.

Dosages for a particular patient can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose can be determined by the efficacy of the particular formulation, and the activity, stability or serum half-life of the ASO employed and the condition of the patient, as well as the body weight or surface area of the patient to be treated. The size of the dose can also be determined by the existence, nature, and extent of any adverse side-effects that accompany the administration of a particular composition in a particular patient.

Optimal precision in achieving effective ASO concentrations within a range yielding maximum efficacy with minimal toxicity may require a regimen based on the kinetics of the pharmaceutical composition's availability to the targeted heart tissues. Distribution, equilibrium, and elimination of a pharmaceutical composition may be considered when determining the optimal concentration for a treatment regimen. Generally, the pharmaceutical compositions of the present invention may be administered in a manner that maximizes efficacy and minimizes toxicity.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See e.g., U.S. Pat. No. 6,747,002, which is entirely expressly incorporated herein by reference.

More specifically, the ASO(s) of the present application may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the ASO(s) of the present application may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.01 μg-30 mg, about 0.01 μg-20 mg or about 0.01-10 mg per day to adults (at about 60 kg). In the case of other animals, the dose calculated for 60 kg may be administered as well.

Doses of ASO(s) of the present application can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 m/ml serum concentration per single or multiple administration or any range, value or fraction thereof.

As a non-limiting example, treatment of humans or animals can be provided as a onetime or periodic dosage of the ASO(s) of the present application 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the ASO(s) of the present application may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the ASO(s) of the present application may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the ASO(s) of the present application may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days. The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the ASO(s) of the present application may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the ASO(s) of the present application may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a weck for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the ASO(s) of the present application may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

### Combination Therapy

In some embodiments, the ASO of the present application can be administered in combination with one or more other therapeutic agents. The ASO of the present application and other therapeutic agents can be administered simultaneously or sequentially by the same or different routes of administration. The determination of the identity and amount of therapeutic agent(s) for use in the methods described herein can be readily made by ordinarily skilled medical practitioners using standard techniques known in the art. Generally, the ASO of the present application is administered in combination with an effective amount of another therapeutic agent that treats cardiac hypertrophy and/or any heart disease or heart disease symptom associated with cardiac hypertrophy.

Other therapeutic agents include, but are not limited to, beta blockers, anti-hypertensives, cardiotonics, anti-thrombotics, vasodilators, hormone antagonists, inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

More specifically, an ASO may be combined with another therapeutic agent including, but not limited to, an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In specific embodiments, the ASO of the present application is combined with an antihyperlipoproteinemic agent including aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof, acifran, azacosterol, benfluorex, β-benzalbutyramide, carnitine, chondroitin sulfate, clomestrone, detaxtran, dextran sulfate sodium, critadenine, furazabol, meglutol, melinamide, mytatrienediol, ornithine, γ-oryzanol, pantethine, pentacrythritol tetraacetate, phenylbutyramide, pirozadil, probucol (lorelco), β-sitosterol, sultosilic acid-piperazine salt, tiadenol, triparanol and xenbucin.

In some embodiments, the ASO of the present application is combined with an antiarteriosclerotic agent such as pyridinol carbamate. In other embodiments, the ASO is combined with an antithrombotic/fibrinolytic agent including, but not limited to anticoagulants (acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin); anticoagulant antagonists, antiplatelet agents (aspirin, a dextran, dipyridamole (persantin), heparin, sulfinpyranone (anturane) and ticlopidine (ticlid)); thrombolytic agents (tissue plaminogen activator (activasc), plasmin, pro-urokinase, urokinase (abbokinasc) streptokinase (streptasc), anistreplase/APSAC (cminase)); thrombolytic agent antagonists or combinations thereof).

In other embodiments, the ASO is combined with a blood coagulant including, but not limited to, thrombolytic agent antagonists (amiocaproic acid (amicar) and tranexamic acid (amstat); antithrombotics (anagrelide, argatroban, cilstazol, daltroban, defibrotide, enoxaparin, fraxiparine, indobufen, lamoparan, ozagrel, picotamide, plafibride, tedelparin, ticlopidine and triflusal); and anticoagulant antagonists (protamine and vitamin K1).

Alternatively, the ASO may be combined with an antiarrhythmic agent including, but not limited to, Class I antiarrythmic agents (sodium channel blockers), Class II antiarrythmic agents (beta-adrenergic blockers), Class III antiarrythmic agents (repolarization prolonging drugs), Class IV antiarrhythmic agents (calcium channel blockers) and miscellaneous antiarrythmic agents. Non-limiting examples of sodium channel blockers include Class IA (disppyramide (norpace), procainamide (pronestyl) and quinidine (quinidex)); Class IB (lidocaine (xylocalne), tocamide (tonocard) and mexiletine (mexitil)); and Class IC antiarrhythmic agents, (encamide (enkaid) and ficcamide (tambocor)).

Non-limiting examples of a beta blocker (also known as a β-adrenergic blocker, a β-adrenergic antagonist or a Class II antiarrhythmic agent) include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfmalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol. Non-limiting examples of an agent that prolongs repolarization, also known as a Class III antiarrhythmic agent, include amiodarone (cordarone) and sotalol (betapace).

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellancous calcium channel blocker such as bencyclane, etafenone, magnesium, mibefradil or perhexyline. In certain embodiments a calcium channel blocker comprises a long-acting dihydropyridine (nifedipine-type) calcium antagonist.

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

In other embodiments, the ASO of the present application ASO is combined with an antihypertensive agent including, but not limited to, alpha/beta blockers (labetalol (normodyne, trandate)), alpha blockers, anti-angiotensin II agents, sympatholytics, beta blockers, calcium channel blockers, vasodilators and miscellaneous antihypertensives.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

Non-limiting examples of anti-angiotension II agents include angiotensin converting enzyme inhibitors and angiotension II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan. Non-limiting examples of a sympatholytic include a centrally acting sympatholytic or a peripherally acting sympatholytic. Non-limiting examples of a centrally acting sympatholytic, also known as a central nervous system (CNS) sympatholytic, include clonidine (catapres), guanabenz (wytensin), guanfacine (tenex) and methyldopa (aldomet). Non-limiting examples of a peripherally acting sympatholytic include a ganglion blocking agent, an adrenergic neuron blocking agent, a β-adrenergic blocking agent or an al-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlonc), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

In certain embodiments, an antihypertensive agent may comprise a vasodilator (e.g., a cerebral vasodilator, a coronary vasodilator or a peripheral vasodilator). In particular embodiments, a vasodilator comprises a coronary vasodilator including, but not limited to, amotriphene, bendazol, benfurodil hemisuccinate, benziodarone, chloracizine, chromonar, clobenfurol, clonitrate, dilazep, dipyridamole, droprenilamine, cfloxate, crythrityl tetranitrane, etafenone, fendiline, floredil, ganglefene, herestrol bis(P-diethylaminocthyl ether), hexobendine, itramin tosylate, khellin, lidoflanine, mannitol hexanitrane, medibazine, nicorglycerin, pentacrythritol tetranitrate, pentrinitrol, perhexylinc, pimcthylline, trapidil, tricromyl, trimctazidine, trolnitrate phosphate and visnadinc.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Non-limiting examples of miscellaneous antihypertensives include ajmaline, y-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketonc thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil. In certain aspects, an antihypertensive may comprise an arylethanolamine derivative (amosulalol, bufuralol, dilevalol, labetalol, pronethalol, sotalol and sulfmalol); a benzothiadiazine derivative (althizide, bendroflumethiazide, benzthiazide, benzylhydrochlorothiazide, buthiazide, chlorothiazide, chlorthalidone, cyclopenthiazide, cyclothiazide, diazoxide, epithiazide, cthiazide, fenquizone, hydrochlorothiazide, hydroflumethizide, methyclothiazide, meticrane, metolazone, paraflutizide, polythizide, tetrachlormethiazide and trichlonnethiazide); a N-carboxyalkyl (peptide/lactam) derivative (alacepril, captopril, cilazapril, delapril, enalapril, enalaprilat, fosinopril, lisinopril, moveltipril, perindopril, quinapril and ramipril); a dihydropyridine derivative (amlodipine, felodipine, isradipine, nicardipine, nifedipine, nilvadipine, nisoldipine and nitrendipine); a guanidine derivative (bethanidine, debrisoquin, guanabenz, guanacline, guanadrel, guanazodine, guancthidine, guanfacine, guanochlor, guanoxabenz and guanoxan); a hydrazines/phthalazinc (budralazine, cadralazinc, dihydralazine, endralazine, hydracarbazine, hydralazine, pheniprazine, pildralazine and todralazinc); an imidazole derivative (clonidine, lofexidine, phentolamine, tiamenidine and tolonidine); a quanternary ammonium compound (azamethonium bromide, chlorisondamine chloride, hexamethonium, pentacynium bis (methylsulfate), pentamethonium bromide, pentolinium tartrate, phenactropinium chloride and trimethidinium methosulfate); a reserpine derivative (bietaserpine, deserpidine, rescinnamine, reserpine and syrosingopine); or a sulfonamide derivative (ambuside, clopamide, faro semide, indapamide, quinethazone, tripamide and xipamide).

In other embodiments, the ASO of the present application is combined with a vasopressor. Vasopressors generally are used to increase blood pressure during shock, which may occur during a surgical procedure. Non-limiting examples of a vasopressor, also known as an antihypotensive include amezinium methyl sulfate, angiotensin amide, dimetofrine, dopamine, etifelmin, etilefrin, gepefrine, metaraminol, midodrine, norepinephrine, pholedrine and synephrine.

In some embodiments, the ASO of the present application is combined with treatment agents for congestive heart failure including, but not limited to, anti-angiotension II agents, afterload-preload reduction treatment (hydralazine (apresoline) and isosorbide dinitrate (isordil, sorbitrate)), diuretics, and inotropic agents.

Non-limiting examples of a diuretic include a thiazide or benzothiadiazine derivative (e.g., althiazide, bendroflumethazide, beizthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urca.

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, cnoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for hypertrophy, vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Materials and Methods

### ASO Formulation and Delivery

Antisense oligonucleotides (ASOs) used in this study are 16-nt long RNA oligonucleotides with phosphodiester internucleoside linkages and 2-O-methyl modifications throughout the entire ASO sequence. These chemical modifications render ASOs to be resistant to nuclease degradation in vivo.

ASO sequences used in these examples include the following ASOs (5′-3′), where “m” indicates a 2′-O-methyl modification, and “o” indicates a phosphodiester or phosphorothioate internucleoside linkage:

Mismatch control ASO:
(SEQ ID NO: 9; used in human and mouse as a control)
CmoCmoAmoGmoGmoAmoUmoUmoCmoAmoAmoCmoCmoUmoAmoCm;
Human GATA4 Type 1 uotASO:
(SEQ ID NO: 10)
GmoCmoUmoGmoAmoAmoUmoUmoCmoAmoCmoCmoCmoCmoAmoGm;
Human GATA4 Type II uotASO:
(SEQ ID NO: 7
AmoCmoGmoUmoAmoUmoUmoAmoAmoAmoUmoCmoCmoAmoGmoCm;
Mouse GATA4 Type II uotASO:
(SEQ ID NO: 8)
AmoCmoGmoAmoAmoUmoUmoAmoAmoAmoUmoCmoCmoAmoGmoCm;
Human TBX5 mORF Type II motASO:
(SEQ ID NO: 28)
CmoUmoCmoGmoUmoCmoUmoGmoCmoGmoUmoCmoGmoGmoCmoCm;
Human TBX5 uORF Type II uotASO:
(SEQ ID NO: 29)
AmoAmoGmoAmoGmoCmoAmoCmoCmoGmoGmoCmoAmoAmoCmoCm;
Human eIF4G2 mORF Type II motASO:
(SEQ ID NO: 30)
UmoGmoCmoAmoAmoUmoCmoGmoCmoAmoCmoUmoCmoUmoCmoCm;
Human GJA1 Type II motASO Met1:
(SEQ ID NO: 31)
CmoGmoCmoUmoCmoCmoAmoGmoUmoCmoAmoCmoCmoCmoAmoUm;
Human GJA1 Type II motASO Met100:
(SEQ ID NO: 32)
UmoUmoUmoCmoUmoCmoUmoUmoCmoCmoUmoUmoUmoCmoGmoCm;
Human GJA1 Type II motASO Met125:
(SEQ ID NO: 33)
AmoAmoUmoCmoUmoGmoCmoUmoUmoCmoAmoAmoGmoUmoGmoCm;
Human GJA1 Type II motASO Met147:
(SEQ ID NO: 34)
CmoAmoGmoCmoAmoAmoCmoCmoCmoCmoCmoCmoUmoCmoGmoCm;
Human NKX2-5 mORF Type II motASO:
(SEQ ID NO: 35)
GmoCmoAmoGmoGmoGmoCmoUmoGmoGmoGmoGmoAmoAmoCmoAmoUm;
Human NKX2-5 uORF Type II uotASO:
(SEQ ID NO: 36)
CmoUmoGmoGmoCmoAmoGmoCmoUmoUmoCmoCmoCmoUmoGmoCm;
and
Human MEF2C mORF Type II motASO:
(SEQ ID NO: 37)
AmoAmoUmoCmoUmoUmoUmoUmoUmoUmoCmoUmoCmoCmoCmoCm.

All ASOs were obtained from Integrated DNA Technologies (IDT, 100 nmoles each) and purified using a desalting column. Analytical ESI-MS confirmed the purity and quality of the ASOs.

To facilitate efficient ASO delivery into murine hearts, nanoparticles (Altogen Biosystems, Cat. No. 5031) were used as ASO carriers for intravenous delivery in mice. In accordance with the nanoparticle use instructions and recommendations from the manufacturer (Altogen Biosystems), ASOs were injected once per week in male WT C57BL/6J mice (10-12 weeks old) at a dose of 2.5 mg/kg body weight (in a volume of 150-200 μl) in both isoproterenol (ISO)-induced cardiac hypertrophy (2 weeks) and transverse aortic constriction (TAC) surgery (8 weeks) models. Negative controls included vehicle treatment for the ISO model and sham surgery for the TAC model. A mismatched control ASO and GATA4 ASO2 (˜100 μg) were each dissolved in ˜150-200 μl RNase-free water. The diluted ASOs were incubated with 50 μl of nanoparticle-based in vivo transfection reagent (Altogen Biosystems, Cat. No. 5031) in sterile tubes for 20 mins at room temperature. Transfection enhancer (10 μl, Altogen Biosystems, Cat. No. 1799) was added to the mixture, vortexed gently, and incubated for 5 mins at room temperature. The nanoparticle-ASO complex was mixed with an appropriate volume of sterile 5% glucose (w/v) solution and delivered into the murine heart by intravenous tail vein injections (after mice are anesthetized using 2.0% isoflurane) once a week for 8 weeks after ISO treatment or TAC surgery.

### Molecular Cloning

WT and mutant firefly luciferase reporters were made based on the backbone plasmid purchased from Addgene (https://www.addgene.org/114670/). 5′UTR DNA fragments were PCR amplified from cDNA prepared from HEK293T or AC16 human cardiomyocyte (CM) cell line using the following primers:

Primer sequences
(without 5′ extra sequence, see below):
GATA4 5′UTR:
FWD:
(SEQ ID NO: 11)
AACGTCTCCACACttggaggcggccggc
REV:
(SEQ ID NO: 12)
AACGTCTCTCTTCCATggtccctgcgagctccc
TBX5 5′UTR:
FWD:
(SEQ ID NO: 48)
AACGTCTCCACACtagttggataggcgatttcagtactttgtgag
REV:
(SEQ ID NO: 49)
AACGTCTCTCTTCCATggtgcgcccagggcc
NKX2-5 5′UTR:
FWD:
(SEQ ID NO: 50)
AACGTCTCCACACgggcggcggcaccttgcag
REV:
(SEQ ID NO: 51)
AACGTCTCTCTTCCATgcccgcgcacccgtc

The forward primers contain the gene-specific 5′UTR sequences in small case with an extra sequence “AACGTCTCCACAC” (in caps; SEQ ID NO:11) at the 5′ end to add a BsmbI site. The reverse primers contain the gene-specific 5′ UTR sequences in small case with an extra sequence “AACGTCTCTCTTCCAT” (in caps; SEQ ID NO:12) at the 5′ end to add a BsmbI site. Each of the amplified DNA fragments and the plasmid backbone were cleaved with BsmbI for 1 hour and then ligated using T4 DNA ligase, followed by selection on X-Gal coated plates.

### Mice

For experiments with WT mice, C57BL/6J mice of the same age (10-12 weeks) and gender (male and female) from littermates or sibling mating were used. All animal procedures were performed in accordance with the National Institutes of Health (NIH) and the University of Rochester Institutional guidelines. All the mice were maintained on a 12-hr light/dark cycle and fed with a normal chow diet at a temperature of 22° C.

### Isoproterenol (ISO) Injection Model

Experimental mice used for this model were siblings generated from intercrosses of WT C57BL/6J mice. Age-matched WT male and female mice at 10-12 weeks of age were subjected to the vehicle (saline) or ISO treatment. ISO or vehicle saline were administered to WT mice daily for 2 weeks using subcutaneous injection (30 mg/kg/day). Excised mouse hearts were flushed with saline to remove the blood, fixed in 10% formalin, and used for histological and immunoblotting analyses.

### Transverse Aortic Constriction (TAC) Surgical Model

Experimental mice used for this model were siblings generated from intercrosses of WT C57BL/6J mice. Age-matched WT male and female mice were subjected to Sham or TAC surgery at 10-12 weeks of age. Each mouse was anesthetized using 2.0% isoflurane, placed on a surgical board with a heating pad (half-inch plexiglass between the animal and the heating pad), and given buprenorphine-SR. A midline cervical incision was made to expose the trachea for visualizing oral intubation using a 22-gauge (PE90) plastic catheter. The catheter was connected to a volume-cycled ventilator supplying supplemental oxygen with a tidal volume of 225-250 μl and a respiratory rate of 120-130 strokes/min. Surgical plane anesthesia was subsequently maintained with 1-1.5% isoflurane.

Procedure for left thoracotomy: Skin was incised, and the chest cavity was opened at the level of the 2nd intercostal space. The transverse section of the aorta was isolated. Transverse aortic constriction was created by placing a (6-0 silk) ligature securely around the trans-aorta and a 27-gauge needle, causing complete occlusion of the aorta. The needle was removed, restoring a lumen with severe stenosis. Lungs were reinflated, and the chest was closed using Vicryl 6-0 suture. Muscle and skin were sutured using a Vicryl 6-0 suture in a running subcuticular pattern. Once the mouse was breathing on its own, it was removed from the ventilator and allowed to recover in a clean cage on a heated pad.

Mice were randomized for experiments using simple randomization with a specific ID number before animal procedures. For group size justification, a power analysis was performed using both G*power 34 version 3.1.9.6 and the function of power.anova.test in R version 3.5.3 (R Foundation for Statistical Computing, Vienna, Austria). The assumptions include the same standard variance in each study group, effect

$$size = \frac{Difference of the means between study groups}{common standard deviation} ,$$

alpha level=0.05, power=0.9, and the number of study groups. The effect size for specific experiments is assumed based on previous similar studies or literature. In previous experiences, a survival rate of ˜90% was observed after the TAC procedure. To offset the possible loss of mice in a treatment group, at least one additional mouse was included per treatment group.

### Echocardiography

Echocardiographic image collection was performed using a Vevo2100 echocardiography machine (VisualSonics, Toronto, Canada) and a linear-array 40 MHz transducer (MS-550D). Image capture was performed in mice under general isoflurane anesthesia with heart rates maintained at 500-550 beats/min. LV systolic and diastolic measurements were captured in M-mode from the parasternal short axis. Fraction shortening (FS) was assessed as follows: % FS=(end diastolic diameter-end systolic diameter)/(end diastolic diameter)×100%. Left ventricular ejection fraction (EF) was measured and averaged in both the parasternal short axis (M-Mode) using the tracing of the end diastolic dimension (EDD) and end systolic dimension (ESD) in the parasternal long axis: % EF=(EDD-ESD)/EDD. Hearts were harvested at multiple endpoints depending on the study.

### Cell Culture and Transfections

Human HEK293T cells were propagated in Dulbecco's modified Eagle's medium (DMEM; Gibco); AC16 adult human ventricle cardiomyocyte cells (SCC109, Sigma) were propagated in an equal mix of F12 and DMEM media. Both media were supplemented with 10% fetal bovine serum, 2 mM L-Glutamine, and 1× penicillin/streptomycin solution. Where specified, cells were transfected with 50 nM siRNA (Thermo Fisher Scientific) or ASOs (IDT), 50 ng plasmid in 96-well assays, or 2,500 ng in 6-well plate assays using Lipofectamine 3000 (Invitrogen). AC16 cells were authenticated and tested for mycoplasma contamination using a detection kit.

### Dual-Luciferase Assay

Untreated HEK293T cells or those depleted of DDX3X via siRNA (50 nM) were seeded in 96-well plates at a density of 1×10⁴ cells per well and left to adhere overnight. The cells were then transfected with an equal amount of experimental FLuc reporter plasmid (50-2,500 ng) and a control Renilla luciferase (RLuc) plasmid (50-2,500 ng) using lipofectamine 3000 for 18 hours. In ASO treatment experiments, 50 nM ASOs were co-transfected with DNA plasmids in HEK293T cells. The cells were then incubated with Dual-Glo luciferase substrate (Promega) according to the manufacturer's recommendations. The final readings of the Fluc were then normalized to Rluc to obtain the relative luminescence reading.

### RNA Purification and RT-qPCR

Media was aspirated from adherent cells and washed twice with chilled PBS. The cells were lysed by adding 1000 μl of Trizol (Qiagen) directly to the cells, then mixed with 200 μl of chloroform and incubated for 5 min on ice. The mixture is then spun down at 16,000 g for 10 min. RNA was then precipitated from the aqueous layer by adding two volumes of isopropanol and spinning down at 16,000×g for 10 min. The pellet was washed twice with 70% ethanol, left to dry, and resuspended in nuclease-free water. For quantitation of mRNA levels, cDNAs were prepared using iScript master mix RT Kit (Biorad) and subsequently qPCR-amplified using SYBR Primer Assay kits (Biorad). Notably, when a primer set was first used, the identity of the resulting PCR product was confirmed by cloning and sequencing. Once confirmed, melting curves were used in each subsequent PCR to verify that each primer set reproducibly and specifically generated the same PCR product.

### Western Blotting

Cells were lysed in RIPA buffer (Thermo Fisher Scientific), and total cell proteins were separated in a 6%-15% denaturing polyacrylamide gel, transferred to polyvinylidene difluoride membranes (PVDF; Amersham Biosciences), and probed using antibodies recognizing GATA4 (ProteinTech), NKX2-5 (ProteinTech), TBX5 (ThermoFisher), MEF2C (Santa Cruz), β-actin (Thermo Fisher Scientific), or DDX3X (Sigma), and then incubated with either a mouse or rabbit secondary antibody conjugated with horseradish peroxidase (GE Biosciences). Blots were quantified using ImageJ (NIH).

### Polysome Profiling and RNA Extraction

1×109 cells were incubated with cycloheximide (100 μg/ml; Sigma) for 10 min and then harvested using a native lysis buffer with 100 mM KCl, 5 mM MgCl2, 10 mM HEPES, pH 7.0, 0.5% Nonidet P-40, 1 mM DTT, 100 U/ml RNasin RNase inhibitor (Promega), 2 mM vanadyl ribonucleoside complexes solution (Sigma-Aldrich (Fluka BioChemika)), 25 μl/ml protease inhibitor cocktail for mammalian tissues (Sigma-Aldrich), cycloheximide (100 ug/ml). The lysate was then spun down at 1,500 g for 5 min to pellet the nuclei. The supernatant was then loaded onto a 10-50% sucrose gradient and spun in an ultracentrifuge at 150,000×g for 2 hours and 20 min. The gradients were then transferred to a fractionator coupled to an ultraviolet absorbance detector that outputs an electronic trace across the gradient. The gradient was then pumped into the fractionator by a 60% sucrose chase solution and divided equally into 12 fractions. For subsequent RNA extraction, 500 μl of each fraction was mixed with an equal amount of chloroform:phenol:isoamyl alcohol and 0.1× volume of 3 M sodium acetate (pH 5.2) then spun down at 16,000×g for 10 min. The upper aqueous layer was mixed with two volumes of 100% ethanol and incubate overnight at −20° C. The solution was then spun at maximum speed for 30 min to pellet the RNA, which was resuspended in 200 μl of water and re-extracted using the same chloroform: phenol: isoamyl alcohol and 0.1× volume of 3 M sodium acetate (pH 5.2) to ensure the complete removal of sucrose. The final pellet was then washed twice with 70% ethanol and resuspended in nuclease-free water.

### Immunofluorescence and Confocal Microscopy

Immunostaining of cells grown on coverglass or chambered slides: AC16 cells and ESC-derived CMs were grown on the coverslips and incubated with 100 nM MitoTracker Red CMXRos (ThermoFisher Scientific) for 30 mins at 37° C. before being fixed for 10 mins with 4% paraformaldehyde in PBS. Cells were washed with PBS for 3×5 mins and permcabilized using ice-cold 0.5% Triton X-100 in PBS for 5 mins. After blocking with 1% BSA in PBS, the coverslips were incubated with indicated primary antibodies (anti-α-actinin 1:1000) in blocking solution (2% BSA in PBS) for 1 hr at RT and then washed with PBS for 3×5 mins. The coverslips were incubated with the Alex Fluor-488 conjugated secondary antibodies (ThermoFisher Scientific, 1:1000) in PBS and washed with PBS for 3×5 mins. Coverslips were air-dried and placed on slides with an antifade mounting medium (containing DAPI). The slides were imaged using an Olympus FV1000 confocal microscope.

### Wheat Germ Agglutinin (WGA) and Phalloidin Staining

WGA (5 mg) was dissolved in 5 ml of PBS (pH 7.4). We performed deparaffinization by following steps: (i) Xylene (100%) for 2×5 mins; (ii) Ethanol (100%) for 2×5 mins; (iii). Ethanol (95%) for 1×5 mins; (iv) ddH₂O for 2×5 mins. The slides were kept in a pressure cooker for 10 min along with citrate buffer (10 mM, pH 6.0) for antigen retrieval. The slides were quenched with 0.1 M glycine in phosphate buffer (pH 7.4) for 1 hour at RT. Circles were made with a Dako pen, and slides were blocked with normal goat serum for 30 min. 10 μg/ml of WGA-Alexa Fluor 488 (Sigma Aldrich) was applied to the slides for a 1 hr incubation at RT. Slides were rinsed in PBST wash buffer 3×5 min followed by PBS for 5 min. A coverslip was placed on the slides with an antifade solution (containing DAPI) for imaging. Five different cross-sectional areas were selected, and cell sizes of at least 500 CM cells were measured per area. For AC16 CM cell cultures, Alexa Fluor™ 594 Phalloidin (Thermo Fisher Scientific) was used to measure cell size following the instruction from the manual. Cultured cells were fixed using a 4% paraformaldehyde in PBS for 10 min, washed with PBS, and permeabilized using 0.2% Triton X-100 for 10 min. Cells were blocked in 2% BSA/PBS for 1 hr and stained with Alexa Fluor™ 594 Phalloidin in 1:1,000 dilution for 30 min at RT. The stained cells were gently washed with PBS for 3×5 min, and the slides were mounted using a mounting medium with DAPI.

### Picrosirius Red Staining

Paraffin-embedded tissue sections were deparaffinized and incubated in a picrosirius red solution (Abcam, Cat. No. ab 150681) at RT for 1 hr. Then, slides were subjected to 2 washes of 1% acetic acid and 100% of ethyl alcohol and then mounted in a resinous medium. Images were captured using the PrimeHisto XE Histology Slide Scanner (Carolina). Six images were selected from each group for analysis. Total collagen content was determined for the whole heart images using the Image J software.

### Statistics

All quantitative data were presented as mean±SEM and analyzed using Prism 8.3.0 software (GraphPad). A Kolmogorov-Smirnov test was used to assess if the data was normally distributed. For comparison between 2 groups, an unpaired Mann-Whitney test for not normally distributed data and an unpaired two-tailed Student t-test for normally distributed data were performed. For multiple comparisons among ≥3 groups, a one-way or two-way ANOVA with Tukey's method for post hoc comparisons and non-parametric Kruskal-Wallis test with Dunn's multiple comparisons were performed. Two-sided P values <0.05 were considered to indicate statistical significance. Specific statistical methods and post hoc tests are described in the figure legends.

### Example 2: Downstream dsRNA Structures Adjacent to uORFs Inhibit mORF Translation

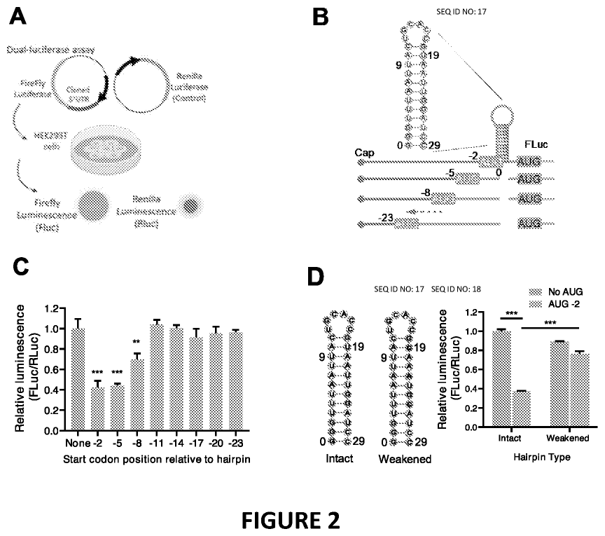

Double-stranded RNA (dsRNA) structures embedded in 5′UTRs have been reported to suppress or enhance translation dependent on its location and structure features. In addition, upstream open reading frames (uORFs) are known to suppress translation of the main open reading frame (mORF). To explore the potential crosstalk between such dsRNA structures and uORFs, artificial luciferase reporters containing dsRNA and uORF elements for dual-luciferase reporter assays were constructed (FIG. 2, Panel A).

Specifically, a series of 5′UTR-firefly luciferase (FLuc) reporter fusions were created from a 5′UTR containing a CA repeat backbone with a stable hairpin Kan-HP1 40-nt away from the 5′-end and 20-nt away from the FLuc ORF start codon. The 5′UTR was synthesized as oligonucleotides (for both + and − strands) from IDT and then cloned into a FLuc construct that corresponds to mORF. The 5′UTR backbone contained a CA repeat (i.e., [CA]*n) which is known to be a linear sequence. In this backbone, the hairpin was added 40-nt away from the 5′ end and 20-nt away from the firefly mORF coding sequence. The hairpin was obtained from the Disney paper because it contained a G at the beginning of it so if an AU is placed before it, it creates a uORF. The AUG was then shifted backward by 3 nucleotides for every reporter up to 27. This backbone was then mutagenized by inserting start codons (i.e., ATG) at various positions spaced by 2 nt up to 23 nt relative to the base of the stem (FIG. 2, Panel B) Dual-Luc assays showed that start codons at positions −2 and −5 confer the most robust suppression of the luciferase activity; weaker inhibition was conferred at position −8 (FIG. 2, Panel C). Start codons at positions −8 to −23 conferred no detectable suppression.

To elucidate the role of hairpin stability on uORF activity, mismatches were introduced into the hairpin of the parent construct (not containing AUG) as well as the one containing a start codon at position −2 (AUG −2) (FIG. 2, Panel D, left). Mutations in the parent sequence confer no suppression over the luciferase activity, whereas mutations in the AUG −2 variant rescued the luciferase activity, suggesting that the RNA structure stability is needed for the AUG codon to confer suppression. Compared to the parent AUG −2 construct, the mutagenized AUG −2 variants exhibited enhanced luciferase activity and protein levels (FIG. 2, Panel D, right). Enhanced luciferase activity was also observed from a parent construct in which the AUG was deleted. Taken together, these results demonstrate a functional connection between upstream ATGs or uORFs, RNA structure stability and translation initiation of mORFs. In particular, a dsRNA stem-loop RNA structure located at a proximal location downstream of uORF (2-11 nt away) was found to enhance uORF activity and reduce mORF translation. These results lend support to the discovery that dsRNA stem-loop structures immediately downstream of uORFs can enhance uORF activity and suppress translation of an mORF.

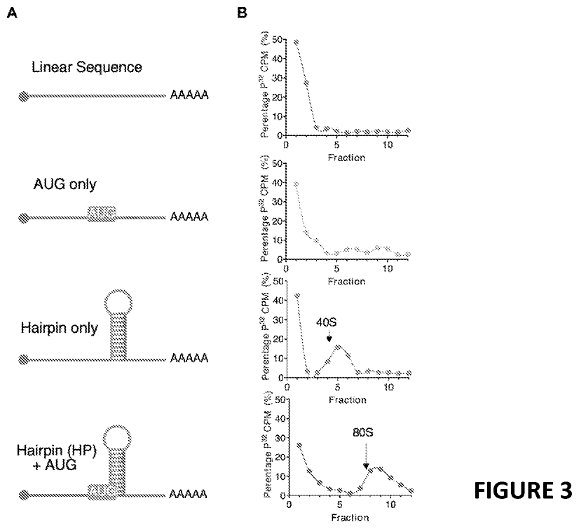

To explore the mechanism underlying the dsRNA-mediated shift of uORF to mORF translation, in vitro transcription of a series of mRNAs with 5′UTRs based on the constructs used in FIG. 2, Panel B, was done and then they were incubated in the rabbit reticulocyte lysate (RRL) (FIA). The lysate was then fractionated on a 10-35% sucrose gradient by ultracentrifugation. The hairpin-bearing 5′UTR of the non-AUG-containing reporter with RRL resulted in co-sedimentation of the 5′UTR with the 40S ribosomal subunit, which was not observed in a control 5′UTR lacking the hairpin and a start codon (FIG. 3, Panel B, in red), suggesting a hairpin-specific co-sedimentation effect (FIG. 3, Panel B, in cyan). Whereas, coupling a start codon with an adjacent downstream hairpin resulted in a shift from the 40S peak in the profile towards an assembled 80S monosome (FIG. 3, Panel B, in green) to a greater extent than a start codon alone (FIG. 3, Panel B, in yellow), suggesting enhanced translation initiation by a hairpin structure downstream of a start codon. Taken together, these results indicate that the presence of a hairpin downstream of a uORF initiation codon enhances the suppressive capability of the uORF against mORF translation, and this synergistic effect between the start codon and the hairpin dsRNA is abolished when the hairpin stem is destabilized.

### Example 3: Presence of uORFs in Human Cardiac Transcriptional Factor mRNAs

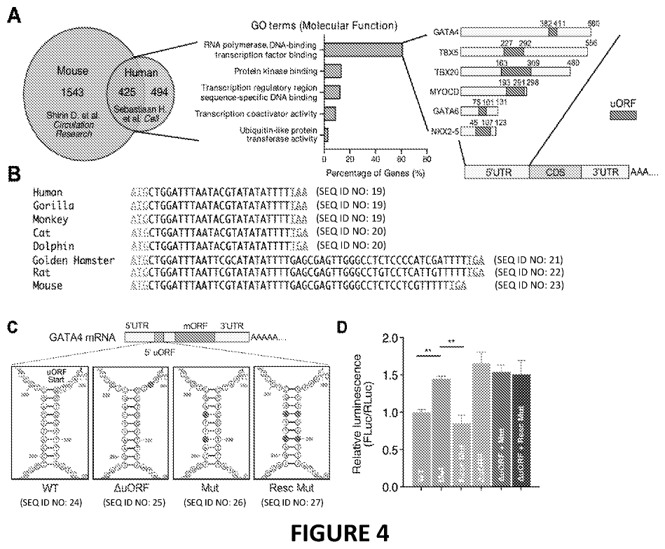

To further investigate a role for dsRNA stem-loop RNA structures and uORFs in suppressing translation of mORFs, a search was conducted to identify naturally existing mRNA transcripts containing one or more uORFs within or surrounding dsRNA structural elements. This was carried out by data mining of unbiased high throughput ribosome profiling (Ribo-seq) databases. Overlapping of Ribo-seq hits uncovered a conserved cohort of mRNAs containing translating uORFs in mice and humans (FIG. 4, Panel A, left, middle). Gene ontology analysis of overlapping genes revealed transcription factors as the top enriched gene set containing translatable uORF, including GATA4, GATA6, TBX5, TBX20, MYOCD, and NKX2-5 (FIG. 4, Panel A, right).

Among these 6 transcription factors, GATA4 was of particular interest, since GATA4 mRNA contains a single uORF exhibiting ribosome footprints in the human heart by Ribo-seq analysis (FIG. 4, Panel A, right) and since the GATA4 uORF is conserved across various mammals and includes an 11-nt sequence downstream of the uORF start codon that is highly conserved through evolution. However, the fact that the uORF protein sequences are not conserved suggests that the GATA4 uORF is more likely to be a regulatory element rather than a bioactive peptide.

GATA4 is a key transcription factor required for cardiomyocyte growth and hypertrophy. RNA structure prediction by the TurboFold tool suggested the presence of a 10 base-pair (bp) stem directly downstream of the uORF start codon shown in the illustration of the predicted structure of 5′UTR (FIG. 4, Panel C). A Selective 2′ Hydroxyl Acylation analyzed by Primer Extension (SHAPE) assay was used to confirm the existence of the double stranded secondary stem structure in the 5′UTR of GATA4 mRNA. In brief, nucleotides located in double-stranded stem structures tend to be less modified by the electrophile, N-methylisatoic anhydride (NAI), while single-stranded regions are exposed for more intense modification. Confirmation of the double-stranded RNA structure was obtained by showing that the predicted 10-bp stem loop downstream of the AUG start codon exhibited the lowest SHAPE activity (data not shown). This result was further evidenced by experiments showing stalling of the 40S ribosome subunit at the hairpin dsRNA region using a toe-printing assay (data not shown).

### Example 4: GATA4 dsRNA Structures Downstream of uORF Start Codon Regulates its Translation Efficiency

GATA4 mRNA is identified as an archetypal transcript containing uORF (FIG. 4, Panels A, B) and dsRNA structural elements (FIG. 4, Panel C) in the 5′UTR. To test whether the uORF in GATA4 mRNA interacts with the downstream dsRNA region to confer suppression to the main open reading frame (mORF) sequence for GATA4 protein, dual-luciferase (Luc) assays were performed in human embryonic kidney cells (HEK293T). The cells were transfected for 48 hr with a test reporter consisting of a cloned uORF-bearing the 5′UTR of GATA4 directly upstream of a firefly Luc (FLuc) ORF together with a control Renilla Luc (RLuc). Relative luminescence (FLuc/RLuc) was then measured using a luminometer. A control uORF-lacking (ΔuORF) reporter was generated by mutating the ATG start codon of the uORF to a TTG codon (i.e., A-to-T mutation) (FIG. 4, Panel C).

Compared to the WT reporter, the ΔuORF reporter exhibited a two-fold increase in Luc activity, consistent with the role of uORF in suppressing translation of the mORF (FIG. 4, Panel D). It was further hypothesized that this suppressive effect could be modulated through interactions with cis-acting RNA secondary structures. Using RNA-structure predictions and SHAPE mapping (not shown), a 10 bp double-stranded region downstream of the uORF start codon (FIG. 4, Panel C) was identified. Together with the WT and ΔuORF mutant, a mutant 5′UTR reporter with mutations in the 10 bp stem (“Mut”), was constructed. Upon their transfection into cells, a marked increase in luciferase activity was observed when disrupting the stability of the double-stranded stem (“Mut”). However, this effect was reversed when appropriate rescuing mutations were introduced back into the stem (see “Resc Mut” in FIG. 4, Panels C, D). This relationship between structure and luciferase activity was not observed in the ΔuORF variant or in ΔuORF variants additionally containing the mutations in “Mut” or “Resc Mut” (FIG. 4, Panel D) suggesting a purely uORF-dependent mechanism.

### Example 5: GATA4-Targeting ASOs Regulate GATA4 mORF Translation Efficiency in Cells

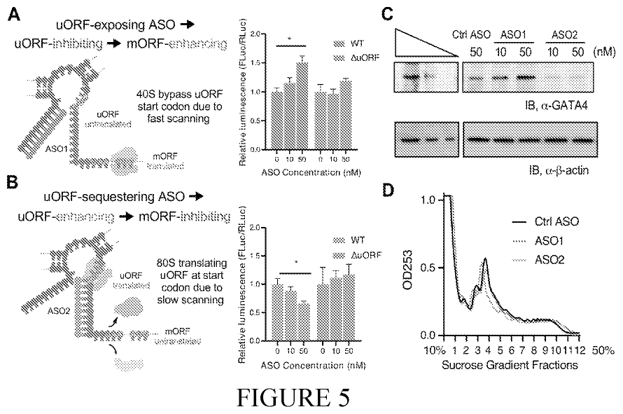

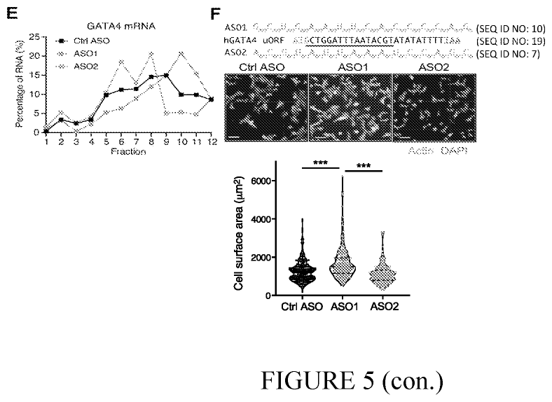

The GATA4 5′UTR variant studies provided an impetus for examining the potential therapeutic effects of using 5′UTR-directed agonists or antagonists to modify GATA4 expression in a therapeutic context. Such studies are predicated on perturbing the activities of the GATA 4 uORF and mORF relative to one another. In this regard, two hypotheses were considered: 1) Disruption of dsRNA structure leads to inactivation of uORF and higher Luc activity; and 2) Sequestration of the uORF results in an increase in its translation, resulting in less Luc activity. The first hypothesis was tested by designing a uORF-suppressing 16-mer ASO (Type I ASO) mimicking the disruption of the upstream strand by preventing it from the sequestering the uORF-containing strand (FIG. 5, Panel A, left). The second hypothesis was tested by designing an uORF-enhancing ASO (Type II uotASO) that can tightly sequester the uORF due to complementary binding, thereby forming a stable 16 bp double-stranded stem (FIG. 5, Panel B, left).

In dual-Luc assays, Type I ASO increased Luc activity, suggesting suppressed uORF translation, while Type II uotASO decreased Luc activity, suggesting enhanced uORF translation (FIG. 5, Panel A, right). These effects are uORF dependent, as indicated by the fact that the ΔuORF reporter activity was unchanged with both ASOs (FIG. 5, Panel B, right).

Targeting the endogenous GATA4 mRNA in AC16 human CMs with these ASOs led to observable protein level changes (FIG. 5, Panel C). The uORF-suppressing Type 1 ASO increased GATA4 protein levels, while the uORF-enhancing Type 2 ASO reduced it. To further confirm the uORF-mediated translational regulation of mORF, polysome profiling was carried out. The results of this analysis showed that the global polysome profiles stayed unchanged (FIG. 5, Panel D). Subsequent RT-qPCR analyses demonstrated that WT 5′UTR-bearing FLuc mRNA shifted to the more heavily translated fractions upon ASO1 treatment while less translatable fractions were obtained upon Type 2 ASO treatment (FIG. 5, Panel E). Inasmuch as no significant changes in mRNA levels were observed (data not shown), it was concluded Type I ASO and Type II uotASO specifically influenced the translation efficiency of the target mRNAs. Thus, when transfected into AC16 cells, Type I ASO caused cardiomyocyte (CM) hypertrophy, while Type II uotASO caused CM atrophy (FIG. 5, Panel F).

### Example 6: GATA4-Targeted Type 2 ASO Blunts Hypertrophic Response in Mouse HF Models

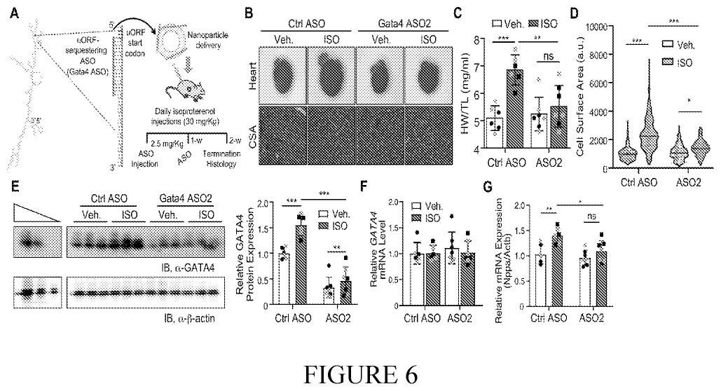

To see whether the in vitro results regarding Type II uotASO-mediated reductions in CM hypertrophy and GATA4 protein expression in FIG. 5 are extrapolatable to in vivo treatment models, WT C57BL/6J mice were injected via the tail vein once a week for two weeks with an analogous mouse-specific GATA4 uORF-enhancing GATA4 Type 2 ASO (2.5 mg/kg; no off-targets as shown from BLAST) formulated in a nanoparticle-based transfection reagent (Altogen Biosystems (FIG. 6, Panel A). After 14 days of isoproterenol (ISO, 30 mg/Kg) injections, the hearts were excised, measured, and histologically analyzed. Mice injected with the mouse GATA4 Type II uotASO were resistant to ISO-induced hypertrophy as evidenced by a significant decrease in the ratio of heart weight (HW) to tibia length (TL) compared to those injected with a control ASO (CASO) (FIG. 6, Panels B, C). Wheat germ agglutinin (WGA) staining of the hearts revealed minimal cellular hypertrophy (FIG. 6, Panels B, D). A consistent reduction in GATA4 protein levels in all mice injected with the therapeutic ASO was observed (FIG. 6, Panel E). Moreover, the GATA4 mRNA level was unchanged following the ASO injections (FIG. 6, Panel F), thereby proving that the observed changes occurred at the level of mRNA translation. Furthermore, consistent with the phenotype of reduced cardiac hypertrophy, a significant reduction of in expression of the critical CM hypertrophy marker Natriuretic Peptide A (ANP) was observed (FIG. 6, Panel G).

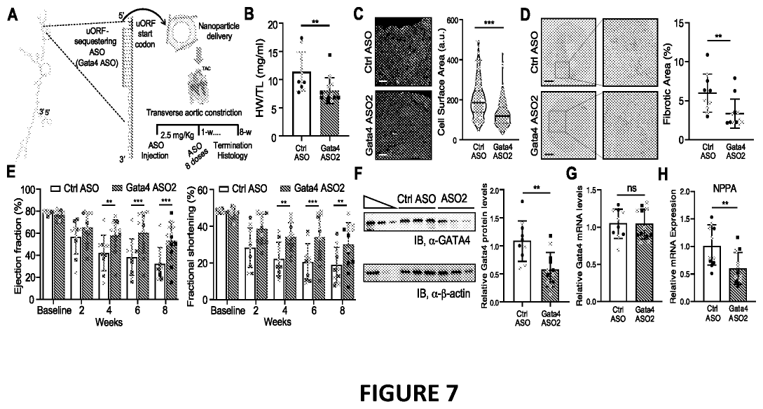

A pressure overload mediated heart failure model was further used to confirm the anti-hypertrophy and cardioprotective effects of the GATA4 uORF-sequestering ASO2 therapeutic. In this case, WT C57BL/6J mice were subjected to transverse aortic constriction surgery to trigger cardiac hypertrophy and heart failure in a 10-weck time course (FIG. 7, Panel A). Heart weight/tibia length (HW/TL) was significantly reduced after treatment with human GATA4 ASO2 (FIG. 7, Panel B). Wheat germ agglutinin (WGA) staining for CM hypertrophy (FIG. 7, Panel C) showed a reduction in CM hypertrophy in mice treated with GATA4 ASO2. Picrosirius red staining for collagen deposition during cardiac fibrosis (FIG. 7, Panel D) showed a reduced fibrosis in mice treated with GATA4 ASO2.

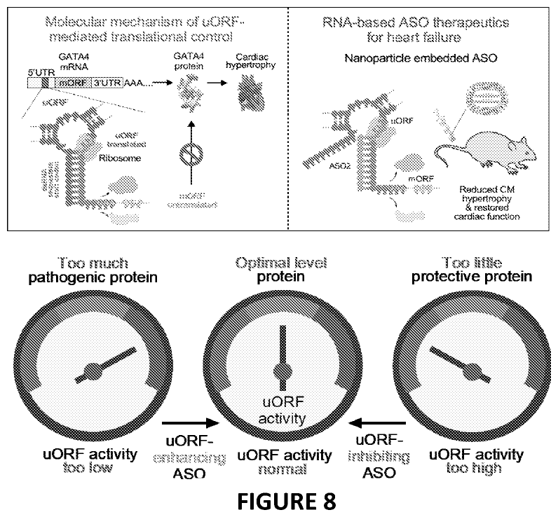

Echocardiography showed that the ejection fraction and fractional shortening were significantly recovered upon treatment with ASO2 (FIG. 7, Panel E). In addition, the therapeutic ASO reduced GATA4 protein levels, while its mRNA expression remained unaltered (FIG. 7, Panels F, G). As expected, a significant reduction in expression of the CM hypertrophy marker ANP was observed (FIG. 7, Panel H). Taken together, the in vitro cell culture and in vivo animal pre-clinical trial data prove the principle of using translation-manipulating ASOs to cither suppress the expression of pathogenic proteins or enhance the expression of protective proteins for disease treatment (FIG. 8).

As an extension to the above experiments, several ΔuORF founder mice carrying an ATG-to-TTG mutation are being created via CRISPR-Cas9 to study the function of GATA4 uORF in vivo.

### Example 7: ASOs Binding Near an mORF Initiation Codon Can Enhance Protein Translation

The above data shows that dsRNA structures embedded in 5′UTRs can enhance or suppress translation depending on its location and associated structure features.

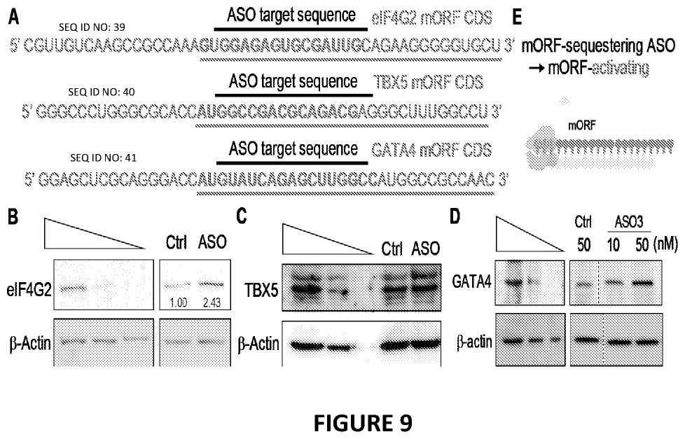

In an effort to further examine mechanisms of translation, it was noted that the translation factor eIF4G2 contains a near-cognate start codon GUG, which is not favorable for translation initiation. To examine the functional consequences of this atypical start codon, a series of 16 nt ASO were synthesized that were designed to target sequences directly downstream of the start codons of several different mORFs as illustrated in FIG. 9, Panel A. More specifically, these ASOs were designed to selectively target sequences directly downstream (indicated by overlining in FIG. 9, Panel A) of the GTG codon in the mORF of cIF4G2, as well as the ATG start codons in the mORFs of GATA4 and the cardiac transcription factor, TBX5. Upon transfection of the foregoing ASOs, Western blot analyses revealed that overexpression of TBX5 or GATA4 protein levels were increases solely after transfection of the foregoing 16 nt ASOs (FIG. 9, Panels B-D).

As used herein, ASOs of this type are termed Type 3 ASOs. In contrast to Type I and Type II uotASOs, the design of a Type 3 ASO does not rely on the presence of an endogenous dsRNA stem structure and can be used to enhance translation of an mORF directly, independent of a uORF. Collectively, these data establish that ASOs can be designed to target sequences downstream of an AUG start codon in an mORF to promote mRNA translation initiation as illustrated in FIG. 9, panel E and FIG. 1, panel C.

Analogous to the targeting strategy using Type I ASOs, this novel targeting strategy can be similarly used to enhance protective and beneficial levels of protein expression in certain disease conditions. This approach is advantageous for therapeutic purposes, since packaging whole genes in a virus, such as adeno-associated viruses (AAV) for gene therapy can be susceptible to size constraints, as well as complications, such as immunogenicity. As a practical application, endogenously overexpressing proteins can potentially be used for targeting GATA4, MEF2C, and TBX5 genes, which promote cardiac regeneration if overexpressed in the heart.

### Example 8: uORF Regulatory Elements in Multiple Cardiac Transcription Factors Support Generic Strategies for Manipulating Translation via Design of Differently Targeted ASOs

As described in Example 3, data mining of theoretical ORFs in 5′UTR regions of human transcriptome (not shown) and Ribo-Seq databases of human and mouse hearts led to the discovery that many cardiac mRNAs contain uORFs (FIG. 4, Panel A). To further examine the impact of uORFs and their associated dsRNA elements, mutations were introduced into the both dsRNA elements and ATG start codon in the uORF of TBX5 (FIG. 9, Panel A). The results of these analyses showed that introduction of such mutations into the ATG start codons of TBX5, MEF2C, and NKX2-5 from to TTG in uORFs (ΔuORF mutants) increased FLuc activity by ˜1.7-3 fold and abolished its dependence on DDX3X (FIG. 9, Panel B and data not shown). DDX3X is a natural trans-acting factor to unwind dsRNA downstream of uORF and inhibit uORF translation. As a consequence, mORF translation can be enhanced. DDX3X controls the biochemical equilibrium and balance of translation between uORF and mORF. The Type I ASO in this application mimics the action of DDX3X to unwind the dsRNA structure. Therefore, this type of ASO can repress uORF translation and promote mORF translation to drive the equilibrium towards translation of mORF.

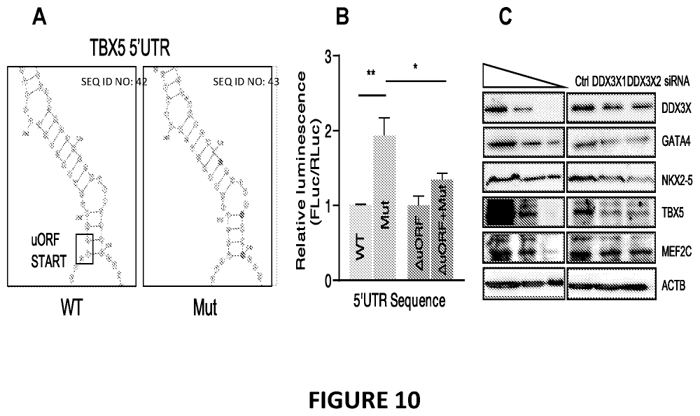

Similar to GATA4, the TBX5 5′UTR region contains a dsRNA element downstream of its uORF. Accordingly, three mutations were introduced to weaken the dsRNA structure to test the impact of this structure element on translational control of mORF (FIG. 10, Panel A). The structural element mutant showed a ˜2-fold increase in FLuc activity (similar to the ΔuORF mutant) compared to the WT reporter (FIG. 10, Panel B, left side). However, mutation of structural dsRNA element on top of ΔuORF did not show a significant change in FLuc activity (FIG. 10, Panel B, right side). Dual Luciferase (DLR) assays with FLuc reporters containing WT 5′UTR of GATA4, MEF2C, TBX5, and NKX2-5 showed that translation and activity of their corresponding FLuc mORF constructs was strongly dependent on DDX3X as shown when transfecting DDX3 directed siRNAs (FIG. 10, Panel C). These results are consistent with the fact, that DDX3X had no effect on the β-actin internal control (FIG. 10, panel C). By contrast, translation and activity of FLuc mORF constructs corresponding to EIF1, EIF5, DENR, and DHX29 were not found to be significantly dependent on DDX3X (not shown). These results suggest that the mechanism of uORF-dsRNA-mediated, DDX3X-regulated translation of GATA4 mORF can be generalizable and appliable to other cardiac transcription factors and potentially many other genes.

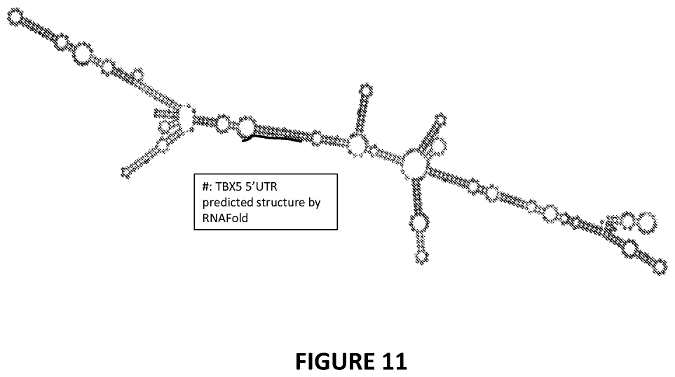

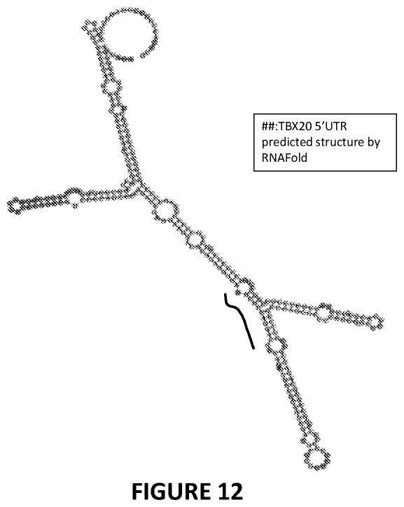

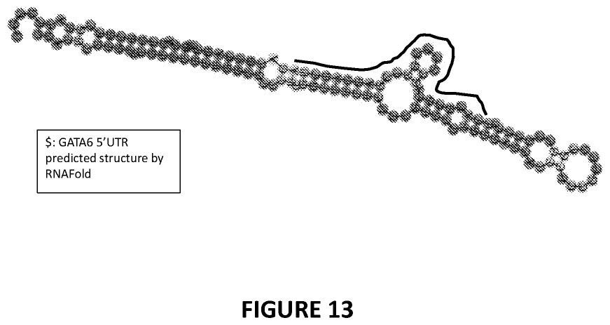

The foregoing experiments establish the function and design of Type I, Type II uotASO and Type II motASOs to selectively increase or decrease translation of a uORF and/or mORF. This has further led to the proposal that the molecular mechanism of dsRNA element-mediated regulation of uORF activity is generalizable as a “translational regulon” that can control mRNA translation. Application of these ASOs to the family of cardiac transcription factors is summarized in Table 1. The information in Table 1 can be used to selectively enhance or suppress translation of the proteins or ORF targets accordingly. FIGS. 11-14 show predicted secondary structure in the 5′UTR of TBX5, TBX20, GATA6 and MYOCD.

TABLE 1

| | | dsRNA | | | |
|---|---|---|---|---|---|
| Gene | ORF target | structure | ASO | Mechanism | Evidence |
| GATA4 | uORF | 10-bp human | Type I uotASO: | Disruption of | See FIG. 5 |
| | | GATA | GₘₒCₘₒUₘₒGₘₒA | dsRNA; | |
| | | | ₘₒAₘₒUₘₒUₘₒCₘₒ | suppression of | |
| | | | AₘₒCₘₒCₘₒCₘₒC | uORF and | |
| | | | ₘₒAₘₒGₘ | activation of | |
| | | | (SEQ ID | mORF | |
| | | | NO: 10) | translation | |
| GATA4 | uORF: | 10-bp human | Type II uotASO: | Formation of | WB in AC16 cells; |
| | ATGCTGGATT | GATA | AₘₒCₘₒGₘₒAₘₒA | artificial dsRNA; | Polysome profiling-RT- |
| | TAATACGTAT | GCUGGAUU | ₘₒUₘₒUₘₒAₘₒAₘ | activation of | qPCR; AC16 cell |
| | ATATTTTTAA | UA | oAₘₒUₘₒCₘₒCₘₒ | uORF and | culture; ISO and TAC- |
| | (SEQ ID | (SEQ ID | AₘₒGₘₒCₘ | suppression of | induced HF mouse |
| | NO: 19) | NO: 3) | (SEQ ID | mORF | models |
| | | | NO: 7) | translation | See FIG. 5 |
| GATA4 | mORF | NA | Type II | Formation of | WB in AC16 cells upon |
| | target: | | motASO: | artificial dsRNA; | ASO3 treatment and |
| | GTATCAGAGC | | GₘₒGₘₒCₘₒCₘₒA | activation of | DDX3X siRNA |
| | TTGGCC | | ₘₒAₘₒGₘₒCₘₒUₘₒ | mORF | knockdown |
| | (SEQ ID | | CₘₒUₘₒGₘₒAₘₒU | translation | See FIG. 5 |
| | NO: 44) | | ₘₒAₘₒCₘ | | |
| | | | (SEQ ID | | |
| | | | NO: 38) | | |
| TBX5 | uORF: | 14-bp human | Type II uotASO | Formation of | WT and AuORF mutant |
| | ATGGTTGCCG | TBX5 uORF: | AₘₒAₘₒGₘₒAₘₒG | artificial | TBX5 5′UTR-bearing |
| | GTGCTCTTGG | GCUUGCCG | ₘₒCₘₒAₘₒCₘₒCₘₒ | dsRNA; | FLuc reporter for DLR |
| | AGGCTATTTT | GUGCUC | GₘₒGₘₒCₘₒAₘₒA | activation of | assays; ASO treatment |
| | GTGTTCTTTG | (SEQ ID | ₘₒCₘₒCₘ | uORF and | experiments |
| | GCGCTTGCCA | NO: 46) | (SEQ ID | suppression of | are ongoing |
| | ACTGGGAAGT | | NO: 29) | mORF | |
| | ATTTAG | | | translation | |
| | (SEQ ID | | | | |
| | NO: 45) | | | | |
| TBX5 | mORF | NA | Type II motASO | Formation of | WB in AC16 |
| | target: | | CₘₒUₘₒCₘₒGₘₒU | artificial | cells upon |
| | TGGCCGACGC | | ₘₒCₘₒUₘₒGₘₒCₘₒ | dsRNA; | DDX3X siRNA KD |
| | AGACGA | | GₘₒUₘₒCₘₒGₘₒG | activation of | |
| | (SEQ ID | | ₘₒCₘₒCₘ | mORF | |
| | NO: 47) | | (SEQ ID | translation | |
| | | | NO: 28) | | |
| NKX2-5 | mORF | NA | Type II motASO | Formation of | ASO treatment |
| | | | GₘₒCₘₒAₘₒGₘₒG | artificial | experiments |
| | | | ₘₒGₘₒCₘₒUₘₒGₘₒ | dsRNA; | are ongoing |
| | | | GₘₒGₘₒGₘₒAₘₒA | activation of | |
| | | | ₘₒCₘₒAₘₒUₘ | mORF | |
| | | | (SEQ ID | translation | |
| | | | NO: 35) | | |
| NKX2-5 | uORF | 4-bp dsRNA | Type II uotASO | Formation of | |
| | | predicted | CₘₒUₘₒGₘₒGₘₒC | artificial dsRNA; | |
| | | in | ₘₒAₘₒGₘₒCₘₒUₘₒ | activation of | |
| | | uORF | UₘₒCₘₒCₘₒCₘₒU | uORF and | |
| | | | ₘₒGₘₒCₘ | suppression of | |
| | | | (SEQ ID | mORF | |
| | | | NO: 36) | translation | |
| MEF2C | mORF | NA | Type II motASO | Formation of | ASO treatment |
| | | | AₘₒAₘₒUₘₒCₘₒU | artificial | experiments |
| | | | ₘₒUₘₒUₘₒUₘₒUₘ | dsRNA; | are ongoing |
| | | | oUₘₒCₘₒUₘₒCₘₒ | activation of | |
| | | | CₘₒCₘₒCₘ | mORF | |
| | | | (SEQ ID | translation | |
| | | | NO: 37) | | |
| MEF2C | uORF | TBD | Type II uotASO | Formation of | ASO treatment |
| | | | UₘₒCₘₒCₘₒAₘₒC | artificial dsRNA; | experiments |
| | | | ₘₒCₘₒUₘₒGₘₒAₘₒ | activation of | are ongoing |
| | | | UₘₒUₘₒCₘₒAₘₒA | uORF and | |
| | | | ₘₒAₘₒCₘ | suppression of | |
| | | | (SEQ ID | mORF | |
| | | | NO: 52) | translation | |
| eIF4G2 | mORF | NA | Type II motASO | Formation of | ASO treatment |
| | | | UₘₒGₘₒCₘₒAₘₒA | artificial dsRNA; | experiments |
| | | | ₘₒUₘₒCₘₒGₘₒCₘₒ | activation of | are ongoing |
| | | | AₘₒCₘₒUₘₒCₘₒ | mORF | |
| | | | UₘₒCₘₒCₘ | translation | |
| | | | (SEQ ID NO: 30) | | |
| GJA1 | mORF & | NA | Type II motASO | Formation of | ASO treatment |
| (Met1) | alternative | | CₘₒGₘₒCₘₒUₘₒC | artificial dsRNA; | experiments |
| | initiation | | ₘₒCₘₒAₘₒGₘₒUₘₒ | activation of | are ongoing |
| | sites | | CₘₒAₘₒCₘₒCₘₒ | mORF | |
| | | | CₘₒAₘₒUₘ | translation | |
| | | | (SEQ ID NO: 31) | | |
| GJA1 | mORF & | NA | Type II motASO | Formation of | ASO treatment |
| (Met100) | alternative | | UₘₒUₘₒUₘₒCₘₒU | artificial dsRNA; | experiments |
| | initiation | | ₘₒCₘₒUₘₒUₘₒCₘₒ | activation of | are ongoing |
| | sites | | CₘₒUₘₒUₘₒUₘₒ | mORF | |
| | | | CₘₒGₘₒCₘ | translation | |
| | | | (SEQ ID NO: 32) | | |
| GJA1 | mORF & | NA | Type II motASO | Formation of | ASO treatment |
| (Met125) | alternative | | AₘₒAₘₒUₘₒCₘₒU | artificial dsRNA; | experiments |
| | initiation | | ₘₒGₘₒCₘₒUₘₒUₘₒ | activation of | are ongoing |
| | sites | | CₘₒAₘₒAₘₒGₘₒUₘₒ | mORF | |
| | | | GₘₒCₘ | translation | |
| | | | (SEQ ID NO: 33) | | |
| GJA1 | mORF & | NA | Type II motASO | Formation of | ASO treatment |
| (Met147) | alternative | | CₘₒAₘₒGₘₒCₘₒA | artificial dsRNA; | experiments |
| | initiation | | ₘₒAₘₒCₘₒCₘₒCₘₒ | activation of | are ongoing |
| | sites | | CₘₒCₘₒCₘₒUₘₒC | mORF | |
| | | | ₘₒGₘₒCₘ | translation | |
| | | | (SEQ ID NO: 34) | | |
| GATA6 | uORF | 10-bp dsRNA | Type II uotASO | Formation of | Not planned yet |
| | | predicted | CₘₒGₘₒGₘₒGₘₒC | artificial dsRNA; | |
| | | in | ₘₒGₘₒGₘₒGₘₒCₘₒ | activation of | |
| | | uORF | GₘₒCₘₒUₘₒGₘₒCₘₒ | uORF and | |
| | | | GₘₒCₘ | suppression of | |
| | | | (SEQ ID NO: 53) | mORF | |
| | | | | translation | |
| TBX20 | uORF | 10-bp dsRNA | Type II uotASO | Formation of | Not planned yet |
| | | predicted | GₘₒAₘₒAₘₒGₘₒG | artificial dsRNA; | |
| | | 14-nt | ₘₒCₘₒGₘₒGₘₒAₘₒ | activation of | |
| | | away from | AₘₒAₘₒCₘₒGₘₒAₘₒ | uORF and | |
| | | uORF AUG | GₘₒCₘ | suppression of | |
| | | (AUG buried | (SEQ ID NO: 54) | mORF | |
| | | in dsRNA) | | translation | |
| MYOCD | uORF | 7-bp dsRNA | Type II uotASO | Formation of | Not planned yet |
| | | predicted | UₘₒAₘₒAₘₒCₘₒA | artificial dsRNA; | |
| | | 3-nt | ₘₒAₘₒCₘₒCₘₒCₘₒ | activation of | |
| | | away from | AₘₒGₘₒAₘₒAₘₒUₘₒ | uORF and | |
| | | uORF AUG | UₘₒCₘ | suppression of | |
| | | (AUG exposed | (SEQ ID | mORF | |
| | | in loop) | NO: 55) | translation | |

### Example 9: ASO-Mediated Modulation of uORF Regulates GATA4 Protein Expression

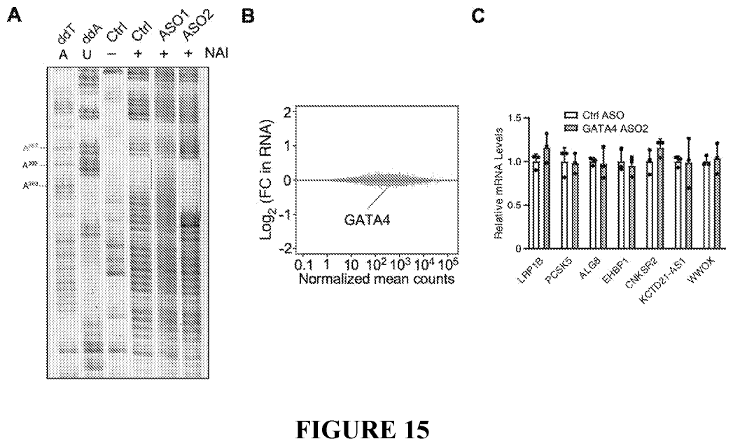

FIG. 15 shows on-target and off-target effects of GATA4-targeting ASOs. In vitro RNA SHAPE analysis of the secondary structure of GATA4 uORF-dsRNA region under ASO1 (SEQ ID NO:10, a type I uotASO), ASO2 (SEQ ID NO:7, a type II uotASO and control ASO (SEQ ID NO:9) treatment showed the disruption caused by ASO1 (Panel A). GATA4-targeting ASO2 does not cause any transcriptome-wide mRNA degradation (Panel B) and does not significantly impact relative mRNA levels of off-target mRNAs (Panel C).

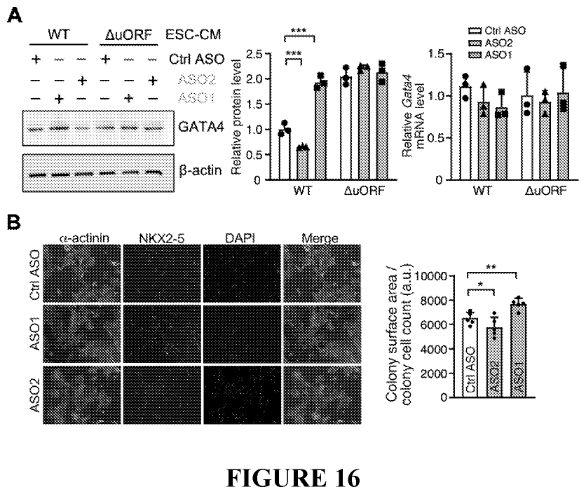

FIG. 16 shows ASO-mediated modulation of uORF regulates GATA4 protein expression and CM hypertrophy in human ESC-derived CMs. As shown in Panel A, GATA4 protein level is increased in ASO1 treated cell and decreased in ASO2-treated cells. Such effect is abolished in cells with mutated uORF. Similarly, Panel B shows increased colony surface area/colony cell count in ASO1 treated cells, and decreased colony surface arca/colony cell count in ASO2 treated cells.

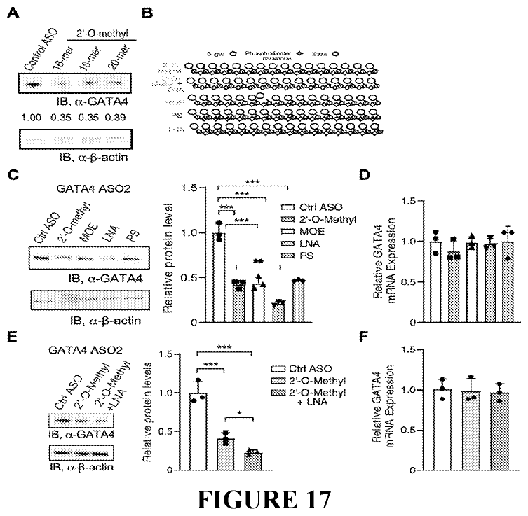

FIG. 17 also shows further that various forms of nucleotide modification of ASO2 can also be used to control ASO-mediated modulation of uORF, which regulates GATA4 protcin expression and CM hypertrophy in human ESC-derived CMs.

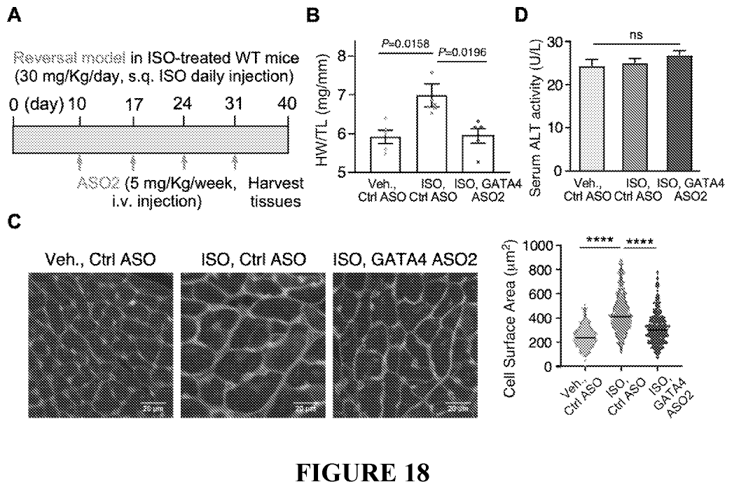

FIG. 18 shows reversal treatment of ISO-induced cardiac hypertrophy model by ASO2. Weckly ASO2 intravenous injection was started after 10 days post-ISO daily subcutaneous injections when cardiac hypertrophy was established (Panel A). Four doses of ASO2 were offered in the ISO model for 40 days (40 ISO injections have strong pro-hypertrophy effects). Both heart weight/tibia length ratio and wheat germ agglutinin staining data showed that ASO2 could reverse cardiac hypertrophy after establishing existing pathological symptoms (Panel B, C). Mice treated with ASO2 had no liver toxicity using an alanine aminotransferase kit (Panel D).

### Example 10: Application of uORF- and mORF-Targeted ASOs for Other mRNAs Encoding Transcription and Translation Factors

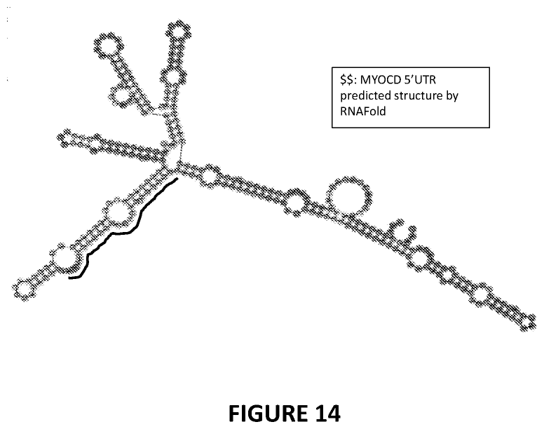

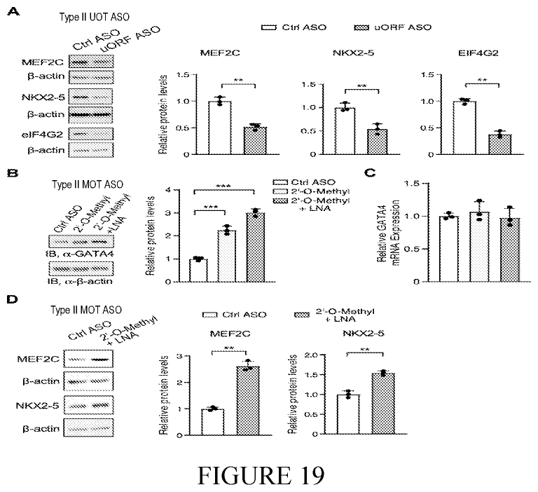

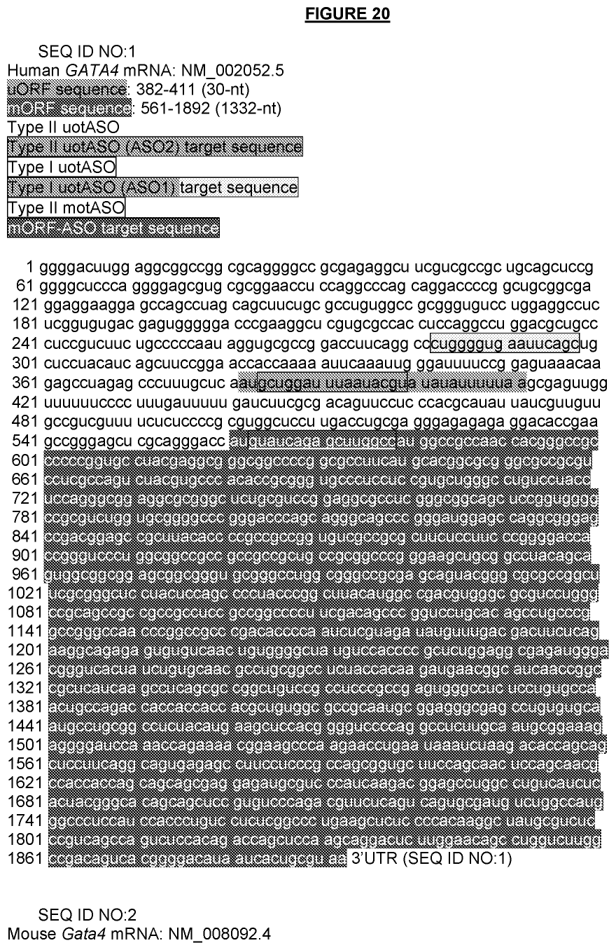

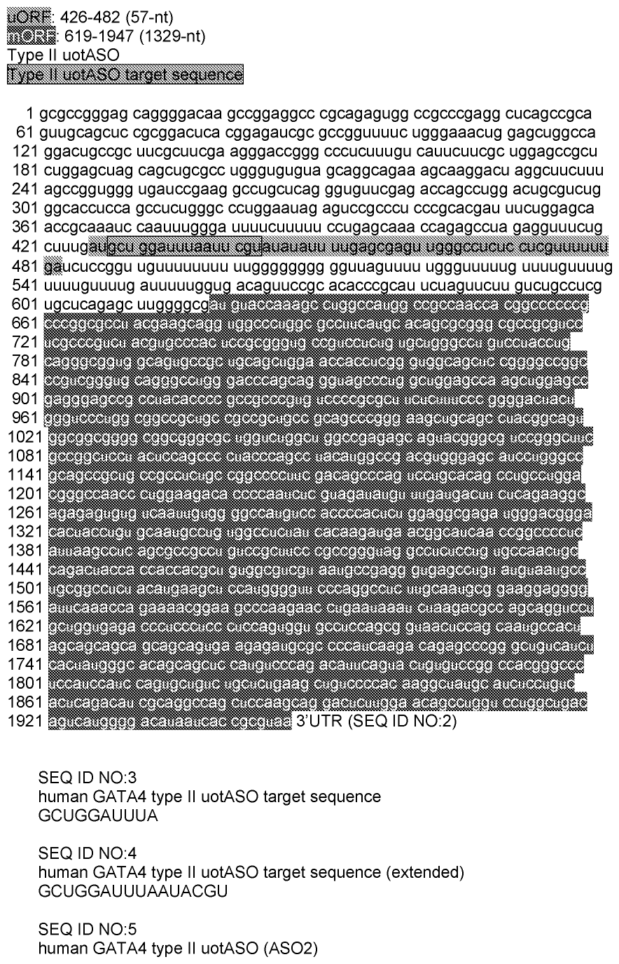

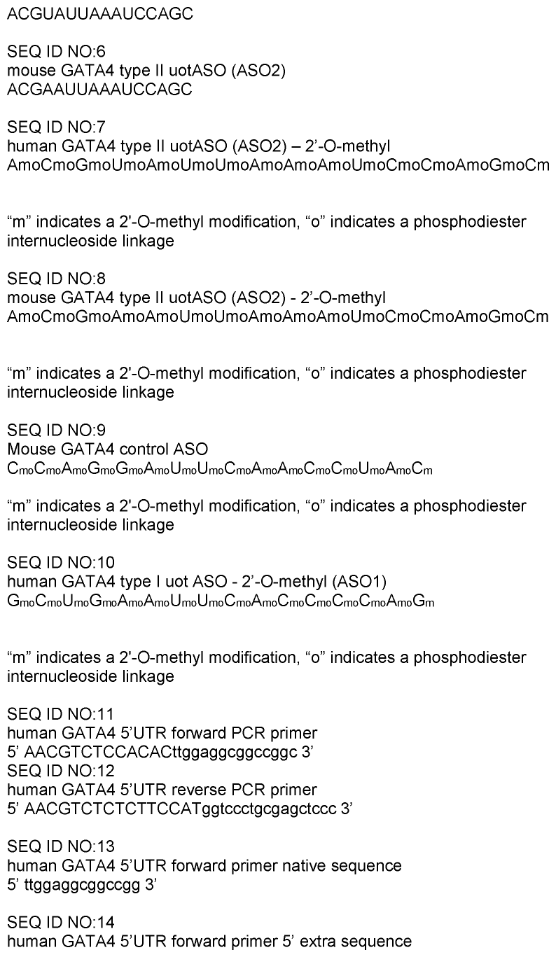

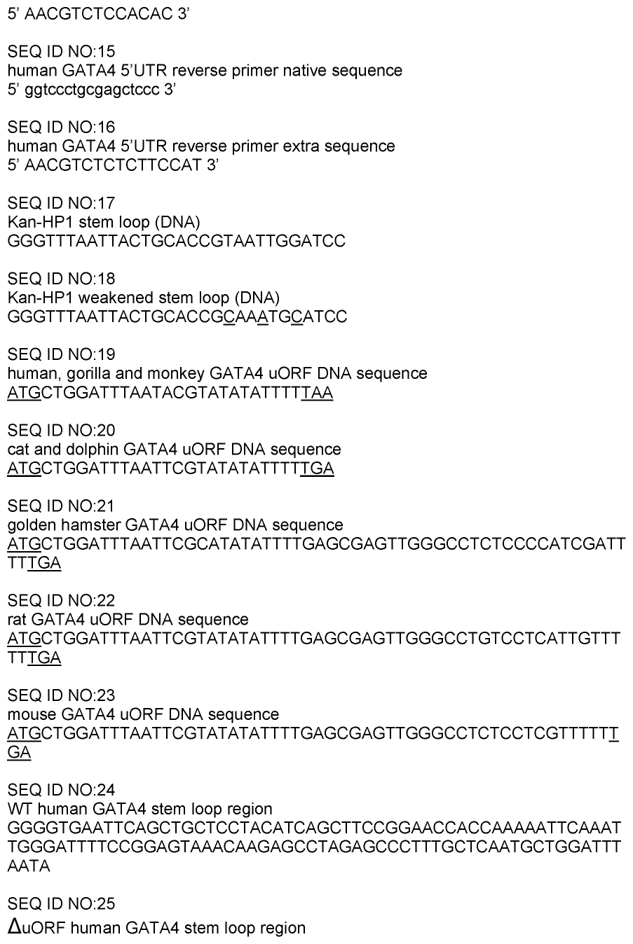

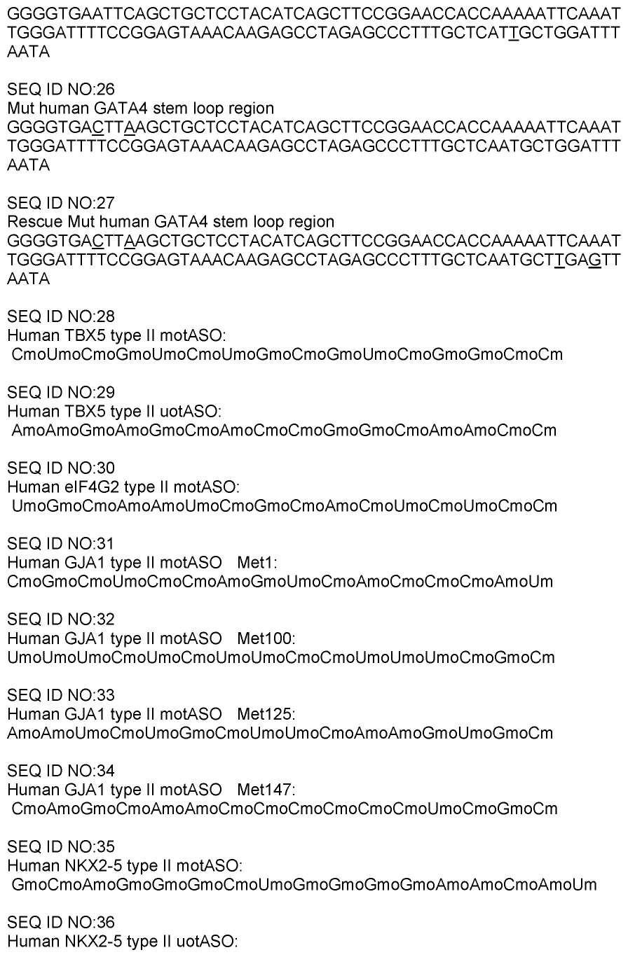

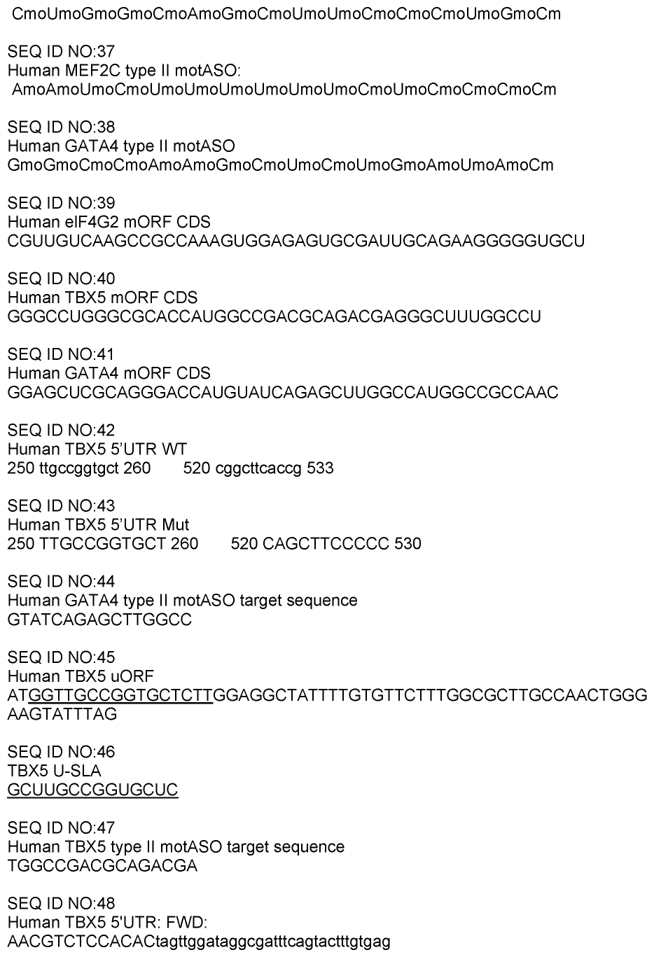

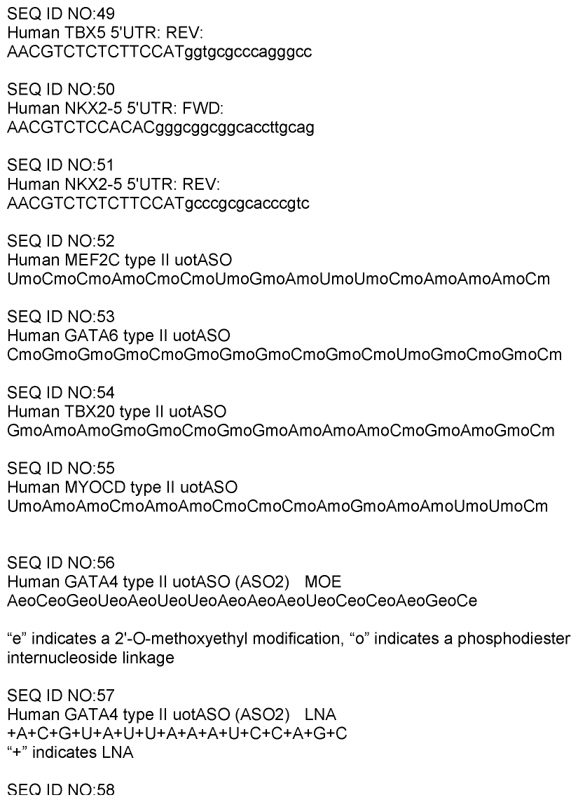

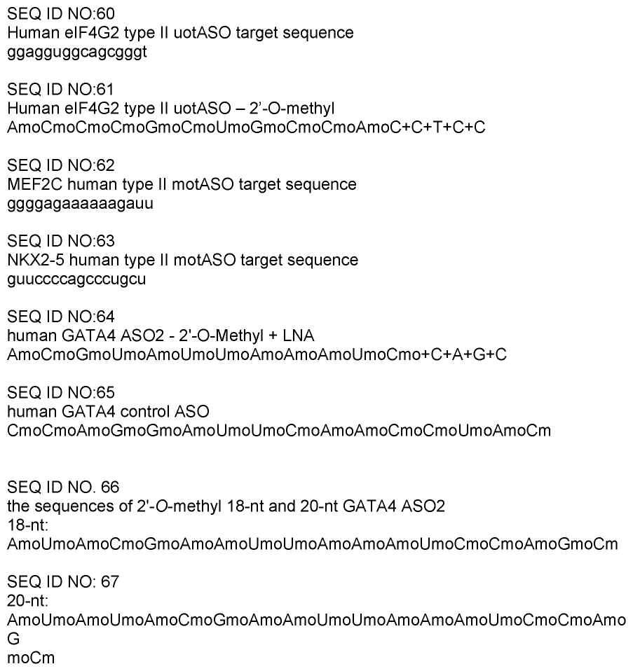

FIG. 19 shows the application of Type II ASOs and Type III ASOs to modulate expression of other proteins and their mRNAs. Type II ASOs can be used to reduce relative protein levels of MEF2C, NKX2-5 and EIF4G2 (FIG. 19A). Type III ASOs can enhance the relative protein levels of GATA4; maximal enhancement is achieved with LNA modifications (FIG. 19B), even though relative mRNA expression is not significantly impacted (FIG. 19C). Type III ASOs can enhance the relative protein levels of MEF2C and NKX2-5 as well (FIG. 14D).

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 7, 2022, is named “1134-095 PCT.xml” and is 114,479 bytes in size. The sequence listing contained in this .XML file is part of the specification and is hereby incorporated by reference herein in its entirety.

1. An antisense oligonucleotide comprising 8-50 nucleotides including one or more modified nucleotides, wherein the antisense oligonucleotide is capable of binding to a target region in an upstream open reading frame (uORF) of a mRNA of a target gene, wherein the target region forms a double-stranded stem structure with a region of the uORF that is downstream of, and adjacent to, the start codon of the uORF, wherein binding of the antisense oligonucleotide to the target region disrupts the double-stranded stem structure of the uORF and enhances translation of a main open reading frame (mORF) downstream of the uORF of the mRNA of the target gene.

2. The antisense oligonucleotide of claim 1, wherein the target region consists of 5 to 30 nucleotides.

3. The antisense oligonucleotide of claim 1, wherein the antisense oligonucleotide comprises a sequence that is at least 80% complementary to the target region.

4. The antisense oligonucleotide of claim 1, wherein the target gene is selected from the group consisting of CRY AB, DACH1, GATA4, HNF4a, MEF2C, MYBPC, NKX2-5, TBX5 and TCF21.

5. The antisense oligonucleotide of claim 1, comprising SEQ ID NO:10.

6. An antisense oligonucleotide comprising 8-50 nucleotides including one or more modified nucleotides, wherein the antisense oligonucleotide is capable of binding to a target region in an upstream open reading frame (uORF) of a mRNA of a target gene, wherein the target region is downstream of, and adjacent to, a start codon of the uORF, wherein binding of the antisense oligonucleotide to the target region forms a double-stranded antisense oligonucleotide/mRNA hybrid structure that inhibits translation of a downstream mORF of the mRNA of the target gene.

7. The antisense oligonucleotide of claim 6, wherein the target region consists of 5 to 30 nucleotides.

8. The antisense oligonucleotide of claim 6, wherein the antisense oligonucleotide comprises a sequence that is at least 80% complementary to the target region.

9. The antisense oligonucleotide of claim 6, wherein the target gene is selected from the group consisting of CRY AB, DACH1, eIF4G2, EPRS, GATA4, HNF4a, MEF2C, MYBPC, MYOCD, NKX2-5, TBX5, TBX20 and TCF21.

10. The antisense oligonucleotide of claim 6, comprising SEQ ID NO:5, 7, 29, 36, 52, 53, 54, 55, 56, 57, 58 or 61.

11. An antisense oligonucleotide comprising 8-50 nucleotides including one or more modified nucleotides, wherein the antisense oligonucleotide is capable of binding to a target region in an main open reading frame (mORF) of a mRNA of a target gene, wherein the target region is downstream of, and adjacent to, a start codon of the mORF, wherein binding of the antisense oligonucleotide to the target region forms a double-stranded antisense oligonucleotide/mRNA hybrid structure that enhances translation of the mORF of the mRNA of the target gene.

12. The antisense oligonucleotide of claim 11, wherein the target region consists of 5 to 30 nucleotides.

13. The antisense oligonucleotide of claim 11, wherein the antisense oligonucleotide comprises a sequence that is at least 80% complementary to the target region.

14. The antisense oligonucleotide of claim 11, wherein the target gene is selected from the group consisting of CRY AB, DACH1, GATA4, HNF4a, MEF2C, MYBPC, NKX2-5, TBX5 and TCF21.

15. The antisense oligonucleotide of claim 1, wherein the antisense oligonucleotide is RNA.

16. The antisense oligonucleotide of claim 1, wherein the modified nucleotides comprise one or more modified sugar moiety.

17. The antisense oligonucleotide of claim 1, wherein the modified nucleotides comprise one or more modified internucleoside linkages.

18. The antisense oligonucleotide of claim 1, further comprising a non-nucleotide conjugation partner.

19. The antisense oligonucleotide of claim 18, wherein the non-nucleotide conjugation partner is a peptide.

20. A pharmaceutical composition, comprising: an antisense oligonucleotide of claim 1; and a pharmaceutically acceptable carrier.

21. A method for treating a disease, comprising: administering to a subject in need of such treatment an effective amount of the pharmaceutical composition of claim 20.


SEQUENCE LISTING (63 sequences)

SEQ ID NO: 1 - RNA, 1892 nt, Homo sapiens
  misc_feature 1..1892: note = Human GATA4 mRNA
  source 1..1892: mol_type = genomic RNA
ggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 2 - RNA, 1947 nt, Mus musculus
  misc_feature 1..1947: note = Mouse Gata4 mRNA
  source 1..1947: mol_type = genomic RNA
gcgccgggagcaggggacaagccggaggcccgcagagtggccgcccgaggctcagccgcagttgcagctccgcggactcacggagatcgcgccggttttctgggaaactggagctggccaggactgccgcttcgcttcgaagggaccgggccctctttgtcattcttcgctggagccgctctggagctagcagctgcgcctgggtgtgtagcaggcagaaagcaaggactaggcttctttagccggtgggtgatccgaaggcctgctcagggtgttcgagaccagcctggactgcgtctgggcacctccagcctctgggccctggaatagagtccgccctcccgcacgatttctggagcaaccgcaaatccaatttgggattttctttttcctgagcaaaccagagcctagaggtttctgctttgatgctggatttaattcgtatatattttgagcgagttgggcctctcctcgttttttgatctccggttgttttttttttggggggggggttagtttttgggtttttgttttgttttgttttgttttgatttttggtgacagttccgcacacccgcattctagttcttgtctgcctcgtgctcagagcttggggcgatgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgtagatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaa

SEQ ID NO: 3 - RNA, 10 nt, Homo sapiens
  misc_feature 1..10: note = human GATA4 type II uotASO target equence
  source 1..10: mol_type = genomic RNA
gctggattta

SEQ ID NO: 4 - RNA, 16 nt, Homo sapiens
  misc_feature 1..16: note = human GATA4 type II uotASO target sequence (extended)
  source 1..16: mol_type = genomic RNA
gctggatttaatacgt

SEQ ID NO: 5 - RNA, 16 nt, Homo sapiens
  misc_feature 1..16: note = human GATA4 type II uotASO (ASO2)
  source 1..16: mol_type = genomic RNA
acgtattaaatccagc

SEQ ID NO: 6 - RNA, 16 nt, unidentified
  misc_feature 1..16: note = mouse GATA4 type II uotASO (ASO2)
  source 1..16: mol_type = unassigned RNA
acgaattaaatccagc

SEQ ID NO: 7 - RNA, 16 nt, synthetic construct
  misc_feature 1..16: note = Synthetic: human GATA4 type II uotASO (ASO2) 2 -O-methyl
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
acgtattaaatccagc

SEQ ID NO: 8 - RNA, 16 nt, synthetic construct
  misc_feature 1..16: note = Synthetic: mouse GATA4 type II uotASO (ASO2) - 2 -O-methyl
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
acgaattaaatccagc

SEQ ID NO: 9 - RNA, 16 nt, synthetic construct
  misc_feature 1..16: note = Synthetic: Mouse GATA4 control ASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
ccaggattcaacctac

SEQ ID NO: 10 - RNA, 16 nt, synthetic construct
  misc_feature 1..16: note = Synthetic: human GATA4 type I uot ASO - 2 -O-methyl (ASO1),
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
gctgaattcaccccag

SEQ ID NO: 11 - DNA, 28 bp, synthetic construct
  misc_feature 1..28: note = Synthetic: human GATA4 5 UTR forward PCR primer
  source 1..28: mol_type = other DNA
aacgtctccacacttggaggcggccggc

SEQ ID NO: 12 - DNA, 33 bp, synthetic construct
  misc_feature 1..33: note = Synthetic: human GATA4 5 UTR reverse PCR primer
  source 1..33: mol_type = other DNA
aacgtctctcttccatggtccctgcgagctccc

SEQ ID NO: 13 - DNA, 14 bp, synthetic construct
  misc_feature 1..14: note = Synthetic: human GATA4 5 UTR forward primer native sequence
  source 1..14: mol_type = other DNA
ttggaggcggccgg

SEQ ID NO: 14 - DNA, 13 bp, synthetic construct
  misc_feature 1..13: note = Synthetic: human GATA4 5 UTR forward primer 5 extra sequence
  source 1..13: mol_type = other DNA
aacgtctccacac

SEQ ID NO: 15 - DNA, 17 bp, synthetic construct
  misc_feature 1..17: note = Synthetic: human GATA4 5 UTR reverse primer native sequence
  source 1..17: mol_type = other DNA
ggtccctgcgagctccc

SEQ ID NO: 16 - DNA, 16 bp, synthetic construct
  misc_feature 1..16: note = Synthetic: human GATA4 5 UTR reverse primer extra sequence
  source 1..16: mol_type = other DNA
aacgtctctcttccat

SEQ ID NO: 17 - DNA, 30 bp, synthetic construct
  misc_feature 1..30: note = Synthetic: Kan-HP1 stem loop (DNA)
  source 1..30: mol_type = other DNA
gggtttaattactgcaccgtaattggatcc

SEQ ID NO: 18 - DNA, 30 bp, synthetic construct
  misc_feature 1..30: note = Synthetic: Kan-HP1 weakened stem loop (DNA)
  source 1..30: mol_type = other DNA
gggtttaattactgcaccgcaaatgcatcc

SEQ ID NO: 19 - DNA, 30 bp, synthetic construct
  misc_feature 1..30: note = Synthetic: human, gorilla and monkey GATA4 uORF DNA sequence
  source 1..30: mol_type = other DNA
atgctggatttaatacgtatatatttttaa

SEQ ID NO: 20 - DNA, 30 bp, synthetic construct
  misc_feature 1..30: note = Synthetic: cat and dolphin GATA4 uORF DNA sequence
  source 1..30: mol_type = other DNA
atgctggatttaattcgtatatatttttga

SEQ ID NO: 21 - DNA, 60 bp, unidentified
  misc_feature 1..60: note = golden hamster GATA4 uORF DNA sequence
  source 1..60: mol_type = unassigned DNA
atgctggatttaattcgcatatattttgagcgagttgggcctctccccatcgatttttga

SEQ ID NO: 22 - DNA, 60 bp, unidentified
  misc_feature 1..60: note = rat GATA4 uORF DNA sequence
  source 1..60: mol_type = unassigned DNA
atgctggatttaattcgtatatattttgagcgagttgggcctgtcctcattgttttttga

SEQ ID NO: 23 - DNA, 57 bp, unidentified
  misc_feature 1..57: note = mouse GATA4 uORF DNA sequence
  source 1..57: mol_type = unassigned DNA
atgctggatttaattcgtatatattttgagcgagttgggcctctcctcgttttttga

SEQ ID NO: 24 - DNA, 112 bp, Homo sapiens
  misc_feature 1..112: note = WT human GATA4 stem loop region
  source 1..112: mol_type = genomic DNA
ggggtgaattcagctgctcctacatcagcttccggaaccaccaaaaattcaaattgggattttccggagtaaacaagagcctagagccctttgctcaatgctggatttaata

SEQ ID NO: 25 - DNA, 112 bp, Homo sapiens
  misc_feature 1..112: note = Delta-uORF human GATA4 stem loop region
  source 1..112: mol_type = genomic DNA
ggggtgaattcagctgctcctacatcagcttccggaaccaccaaaaattcaaattgggattttccggagtaaacaagagcctagagccctttgctcattgctggatttaata

SEQ ID NO: 26 - DNA, 112 bp, synthetic construct
  misc_feature 1..112: note = Synthetic: Mut human GATA4 stem loop region
  source 1..112: mol_type = other DNA
ggggtgacttaagctgctcctacatcagcttccggaaccaccaaaaattcaaattgggattttccggagtaaacaagagcctagagccctttgctcaatgctggatttaata

SEQ ID NO: 27 - DNA, 112 bp, synthetic construct
  misc_feature 1..112: note = Synthetic: Rescue Mut human GATA4 stem loop region
  source 1..112: mol_type = other DNA
ggggtgacttaagctgctcctacatcagcttccggaaccaccaaaaattcaaattgggattttccggagtaaacaagagcctagagccctttgctcaatgcttgagttaata

SEQ ID NO: 28 - RNA, 16 nt, synthetic construct
  misc_feature 1..16: note = Human TBX5 type II motASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
ctcgtctgcgtcggcc

SEQ ID NO: 29 - RNA, 16 nt, synthetic construct
  misc_feature 1..16: note = Human TBX5 type II uotASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
aagagcaccggcaacc

SEQ ID NO: 30 - RNA, 16 nt, synthetic construct
  misc_feature 1..16: note = Human eIF4G2 type II motASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
tgcaatcgcactctcc

SEQ ID NO: 31 - RNA, 16 nt, synthetic construct
  misc_feature 1..16: note = Human GJA1 type II motASO - Met1
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
cgctccagtcacccat

SEQ ID NO: 32 - RNA, 16 nt, synthetic construct
  misc_feature 1..16: note = Human GJA1 type II motASO - Met100
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
tttctcttcctttcgc

SEQ ID NO: 33 - RNA, 16 nt, synthetic construct
  misc_feature 1..16: note = Human GJA1 type II motASO - Met125
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
aatctgcttcaagtgc

SEQ ID NO: 34 - RNA, 16 nt, synthetic construct
  misc_feature 1..16: note = Human GJA1 type II motASO - Met147
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
cagcaacccccctcgc

SEQ ID NO: 35 - RNA, 17 nt, synthetic construct
  misc_feature 1..17: note = Human NKX2-5 type II motASO
  modified_base 1..15: mod_base = OTHER; note = 2'-O-methyl modification and phosphodiester internucleoside linkage
  modified_base 16: mod_base = OTHER; note = 2'-O-methyl modification
  source 1..17: mol_type = other RNA
gcagggctggggaacat

SEQ ID NO: 36 - RNA, 16 nt, synthetic construct
  misc_feature 1..16: note = Human NKX2-5 type II uotASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
ctggcagcttccctgc

SEQ ID NO: 37 - RNA, 16 nt, synthetic construct
  misc_feature 1..16: note = Human MEF2C type II motASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
aatcttttttctcccc

SEQ ID NO: 38 - RNA, 16 nt, synthetic construct
  misc_feature 1..16: note = Human GATA4 type II motASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
ggccaagctctgatac

SEQ ID NO: 39 - RNA, 48 nt, Homo sapiens
  misc_feature 1..48: note = Human elF4G2 mORF CDS
  source 1..48: mol_type = genomic RNA
cgttgtcaagccgccaaagtggagagtgcgattgcagaagggggtgct

SEQ ID NO: 40 - RNA, 44 nt, Homo sapiens
  misc_feature 1..44: note = Human TBX5 mORF CDS
  source 1..44: mol_type = genomic RNA
gggcctgggcgcaccatggccgacgcagacgagggctttggcct

SEQ ID NO: 41 - RNA, 46 nt, Homo sapiens
  misc_feature 1..46: note = Human GATA4 mORF CDS
  source 1..46: mol_type = genomic RNA
ggagctcgcagggaccatgtatcagagcttggccatggccgccaac

SEQ ID NO: 42 - DNA, 22 bp, Homo sapiens
  stem_loop 11..12
  misc_feature 1..22: note = Human TBX5 5 UTR WT
  source 1..22: mol_type = genomic DNA
ttgccggtgctcggcttcaccg

SEQ ID NO: 43 - DNA, 22 bp, Homo sapiens
  stem_loop 11..12
  misc_feature 1..22: note = Human TBX5 5 UTR Mut
  source 1..22: mol_type = genomic DNA
ttgccggtgctcagcttccccc

SEQ ID NO: 44 - DNA, 16 bp, Homo sapiens
  misc_feature 1..16: note = Human GATA4 type II motASO target sequence
  source 1..16: mol_type = genomic DNA
gtatcagagcttggcc

SEQ ID NO: 45 - DNA, 66 bp, Homo sapiens
  misc_feature 1..66: note = Human TBX5 uORF
  source 1..66: mol_type = genomic DNA
atggttgccggtgctcttggaggctattttgtgttctttggcgcttgccaactgggaagtatttag

SEQ ID NO: 46 - RNA, 14 nt, synthetic construct
  misc_feature 1..14: note = Synthetic: TBX5 U-SLA
  source 1..14: mol_type = other RNA
gcttgccggtgctc

SEQ ID NO: 47 - DNA, 16 bp, Homo sapiens
  misc_feature 1..16: note = Human TBX5 type II motASO target sequence
  source 1..16: mol_type = genomic DNA
tggccgacgcagacga

SEQ ID NO: 48 - DNA, 45 bp, synthetic construct
  misc_feature 1..45: note = Synthetic: Human TBX5 5'UTR FWD
  source 1..45: mol_type = other DNA
aacgtctccacactagttggataggcgatttcagtactttgtgag

SEQ ID NO: 49 - DNA, 31 bp, synthetic construct
  misc_feature 1..31: note = Synthetic: Human TBX5 5'UTR REV
  source 1..31: mol_type = other DNA
aacgtctctcttccatggtgcgcccagggcc

SEQ ID NO: 50 - DNA, 32 bp, synthetic construct
  misc_feature 1..32: note = Synthetic: Human NKX2-5 5'UTR FWD
  source 1..32: mol_type = other DNA
aacgtctccacacgggcggcggcaccttgcag

SEQ ID NO: 51 - DNA, 31 bp, synthetic construct
  misc_feature 1..31: note = Synthetic: Human NKX2-5 5'UTR REV
  source 1..31: mol_type = other DNA
aacgtctctcttccatgcccgcgcacccgtc

SEQ ID NO: 52 - RNA, 16 nt, synthetic construct
  misc_feature 1..16: note = Human MEF2C type II uotASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
tccacctgattcaaac

SEQ ID NO: 53 - RNA, 16 nt, synthetic construct
  misc_feature 1..16: note = Human GATA6 type II uotASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
cgggcgggcgctgcgc

SEQ ID NO: 54 - RNA, 16 nt, synthetic construct
  misc_feature 1..16: note = Human TBX20 type II uotASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
gaaggcggaaacgagc

SEQ ID NO: 55 - RNA, 16 nt, synthetic construct
  misc_feature 1..16: note = Human MYOCD type II uotASO
  modified_base 1..15: mod_base = OTHER; note = 2'-O-methyl modification and phosphodiester internucleoside linkage
  modified_base 16: mod_base = OTHER; note = 2'-O-methyl modification
  source 1..16: mol_type = other RNA
taacaacccagaattc

SEQ ID NO: 56 - RNA, 16 nt, synthetic construct
  misc_feature 1..16: note = Synthetic: Human GATA4 type II uotASO (ASO2) - MOE
  modified_base 1..15: mod_base = OTHER; note = 2'-O-methoxyethyl modification and a phosphodiester internucleoside linkage
  modified_base 16: mod_base = OTHER; note = 2'-O-methoxyethyl modification
  source 1..16: mol_type = other RNA
acgtattaaatccagc

SEQ ID NO: 57 - RNA, 16 nt, synthetic construct
  misc_feature 1..16: note = Synthetic: Human GATA4 type II uotASO (ASO2) - LNA
  modified_base 1..16: mod_base = OTHER; note = LNA
  source 1..16: mol_type = other RNA
acgtattaaatccagc

SEQ ID NO: 58 - RNA, 16 nt, synthetic construct
  misc_feature 1..16: note = Synthetic: Human GATA4 type II uotASO (ASO2) - PS
  modified_base 1..3: mod_base = OTHER; note = 2'-O-methyl modification and phosphorothioate internucleoside linkage
  modified_base 4..12: mod_base = OTHER; note = 2'-O-methyl modification and phosphodiester internucleoside linkage
  modified_base 13..15: mod_base = OTHER; note = 2'-O-methyl modification and phosphorothioate internucleoside linkage
  modified_base 16: mod_base = OTHER; note = 2'-O-methyl modification
  source 1..16: mol_type = other RNA
gctgaattcaccccag

SEQ ID NO: 59 - RNA, 16 nt, Homo sapiens
  misc_feature 1..16: note = Human GATA4 type I uotASO (ASO1) target sequence
  source 1..16: mol_type = genomic RNA
ctggggtgaattcagc

SEQ ID NO: 60 - RNA, 16 nt, Homo sapiens
  modified_base 16: mod_base = OTHER; note = thymine
  misc_feature 1..16: note = Human eIF4G2 type II uotASO target sequence
  source 1..16: mol_type = genomic RNA
ggaggtggcagcgggt

SEQ ID NO: 61 - RNA, 16 nt, synthetic construct
  modified_base 14: mod_base = OTHER; note = thymine
  misc_feature 1..16: note = Synthetic: Human eIF4G2 type II uotASO 2 -O-methyl
  source 1..16: mol_type = other RNA
  modified_base 1..11: mod_base = OTHER; note = 2'-O-methyl modification and phosphodiester internucleoside linkage
  modified_base 13..16: mod_base = OTHER; note = LNA
acccgctgccacctcc

SEQ ID NO: 62 - RNA, 16 nt, Homo sapiens
  misc_feature 1..16: note = MEF2C human type II motASO target sequence
  source 1..16: mol_type = genomic RNA
ggggagaaaaaagatt

SEQ ID NO: 63 - RNA, 16 nt, Homo sapiens
  misc_feature 1..16: note = NKX2-5 human type II motASO target sequence
  source 1..16: mol_type = genomic RNA
gttccccagccctgct